SEQUENCE LISTING (18 sequences)

SEQ ID NO: 1 - protein, 18 aa, Macaca mulatta
GFCRCLCRRGVCRCICTR

SEQ ID NO: 2 - protein, 18 aa, Macaca mulatta
GVCRCLCRRGVCRCLCRR

SEQ ID NO: 3 - protein, 18 aa, Macaca mulatta
GFCRCICTRGFCRCICTR

SEQ ID NO: 4 - protein, 18 aa, Macaca mulatta
GICRCICTRGFCRCICVL

SEQ ID NO: 5 - protein, 18 aa, Macaca mulatta
GICRCLCRRGVCRCICVL

SEQ ID NO: 6 - protein, 18 aa, Macaca mulatta
GICRCICVLGICRCICVL

SEQ ID NO: 7 - protein, 18 aa, Papio anubis
CVCRRGVCRCVCTRGFCR

SEQ ID NO: 8 - protein, 18 aa, Papio anubis
CVCRRGVCRCVCRRGVCR

SEQ ID NO: 9 - protein, 18 aa, Papio anubis
CICLLGICRCVCTRGFCR

SEQ ID NO: 10 - protein, 18 aa, Papio anubis
CICLLGICRCVCRRGVCR

SEQ ID NO: 11 - protein, 18 aa, Papio anubis
CICLLGICRCVCRRGVCR

SEQ ID NO: 12 - protein, 18 aa, Papio anubis
CICLLGICRCICLLGICR

SEQ ID NO: 13 - protein, 18 aa, Papio anubis
CFCRRGVCRCVCTRGFCR

SEQ ID NO: 14 - protein, 18 aa, Papio anubis
CFCRRGVCRCVCRRGVCR

SEQ ID NO: 15 - protein, 18 aa, Papio anubis
CFCRRGVCRCICLLGICR

SEQ ID NO: 16 - protein, 18 aa, Papio anubis
CFCRRGVCRCFCFFGVCR

SEQ ID NO: 17 - DNA, 20 bp, Artificial Sequence
  SARS-CoV-2 Nucleocapsid phosphoprotein (N) gene forward primer
atgctgcaatcgtgctacaa

SEQ ID NO: 18 - DNA, 20 bp, Artificial Sequence
  SARS-CoV-2 Nucleocapsid phosphoprotein (N) gene reverse primer
cctctgctcccttctgcgta

(19) United States
(12) Patent Application Publication
(10) Pub. No.: US 2024/0108702 A1
(43) Pub. Date: Apr. 4, 2024

(54) Use Of Naturally Occurring Cyclic Peptides For Treatment Of SARS-COV-2 Infection

(71) Applicant: The University of Southern California, Los Angeles, CA (US)

(72) Inventors: Michael E. Selsted, Pasadena, CA (US); Dat Q. Tran, Alhambra, CA (US); Patti Tran, Alhambra, CA (US); Justin B. Schaal, Orange, CA (US); Virginia Basso, Covina, CA (US); Prasad Tongaonkar, Arcadia, CA (US)

(21) Appl. No.: 18/273,181

(22) PCT Filed: Jan. 12, 2022
(86) PCT No.: PCT/US22/12174

§ 371 Date: Jul. 19, 2023

Related U.S. Application Data
(60) Provisional application No. 63/139,652, filed on Jan. 20, 2021.

(51) Int. Cl. A61K 38/55; A61P 31/14

(52) U.S. Cl. CPC A61K 38/55; A61P 31/14

(57) ABSTRACT

Methods for treating and/or SARS-CoV-2 infection are described that inhibit viral replication, multiple protease activities, and provide anti-inflammatory activity. Such activities can be provided by a single species, such as a naturally occurring cyclic peptide.

This application claims the benefit of U.S. Provisional Patent Application No. 63/139,652 filed on Jan. 20, 2021. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### GOVERNMENT SUPPORT CLAUSE

This invention was made with government support under Grant Nos. AI142959, AI125141, and AI02293 1, awarded by the National Institutes of Health (NIH). The government has certain rights in the invention.

### FIELD

The field is methods for treatment of viral infections, particularly coronavirus infections.

### BACKGROUND

SARS-CoV-2 infection (COVID-19) in its most severe form causes severe acute respiratory distress syndrome (ARDS). Although the mechanisms of COVID-19-induced lung injury are still being elucidated, cytokine release has been implicated in its pathophysiology. The evidence has shown that severely ill COVID-19 patients with ARDS tend to have a high concentration of pro-inflammatory cytokines, such as interleukin (IL)-6, compared to those who are moderately ill. A high level of cytokines also indicates a poor prognosis in COVID-19. Additionally, excessive infiltration of pro-inflammatory cells, mainly involving macrophages and T-helper cells, has been found in lung tissues of patients with COVID-19 related ARDS in postmortem examination. Recently, more studies indicate that cytokine release contributes to the mortality of COVID-19. It appears that severe COVID-19 disease selectively induces a high level of IL-6 systemically and results in the exhaustion of lymphocytes. There is an unmet need to prevent and counteract cytokine release in COVID-19 patients.

θ-Defensins are expressed in Old World monkeys (e.g., macaques and baboons), and are the only known cyclic peptides in animals. The basic θ-defensin backbone structure is produced by head-to-tail splicing of two nonapeptide precursors. In rhesus macaques alternate binary splicing of nonapeptides encoded by three precursor genes provides six θ-defensin isoforms, rhesus theta-defensins RTD-1 to RTD-6. In baboons, alternate nonapeptide splicing produces ten θ-defensin isoforms, baboon theta defensins BTD-1 to BTD-10. θ-defensins are expressed at high levels in granules of neutrophils and in monocytes of these species. These θ-defensins play a major role in the antimicrobial activities of rhesus neutrophil granule extracts. The RTD-1 isoform is the most abundant θ-defensin in macaques, constituting approximately 55% of the total θ-defensin content of rhesus neutrophils.

Humans and other hominids lack θ-defensins due to the presence of a stop codon mutation in the prepro-coding sequence of θ-defensin genes in these species. It has been suggested that the expression of θ-defensins in Old World monkeys is related to differences in immune and inflammatory responses of these nonhuman primates from those of humans.

While α-, β-, and θ-defensins were initially identified on the basis of broad spectrum antimicrobial properties, subsequent studies have disclosed different and distinct immune regulatory roles. For example, some α- and β-defensins are chemotactic for T cells, neutrophils, dendritic cells, and monocytes, and induce secretion of proinflammatory cytokines from activated dendritic cells, peripheral blood mononuclear cells and epithelial cells. It should be appreciated that such pro-inflammatory activity is highly undesirable in the treatment of diseases characterized by severe respiratory distress, such as COVID-19.

The use of a rhesus θdefensin 1 (RTD-1) in a murine model of prophylaxis of SARS via intranasal administration has been described. Unfortunately, doses of this defensin that were effective in improving survival resulted in significant irritation and the formation of lesions at the site of administration. In addition, no direct SARS coronavirus antiviral activity was found. In addition, intravenous administration was found to be ineffective.

Thus, there is still a need for safe and effective compositions and methods for the treatment and/or prevention of coronavirus infections, including SARS-CoV-2, and their sequelae

The following description includes information that may be useful in understanding the present disclosure. It is not an admission that any of the information provided herein is prior art or relevant to the disclosure, or that any publication specifically or implicitly referenced is prior art.

### SUMMARY

Provided herein are compositions and methods that provide both protease inhibition and anti-inflammatory activities effective in treating and/or preventing coronavirus infection, in particular betacoronavirus infection. A stable and non-immunogenic peptide drug is provided that inhibits a range of protease activities, as well as providing an anti-inflammatory activity. The peptide can be applied topically to the respiratory system using an aerosol delivery system. Such an aerosol delivery system can include propylene glycol. Alternatively, the peptide can be delivered by injection and/or infusion.

The peptides and compositions provided herein possess anti-protease and anti-inflammatory properties, and at least one of these (Peptide 1) inhibits SARS-CoV-2 viral replication which correlates with the ability of Peptide 1 to inhibit TMPRSS2 activity. The antiviral, antiprotease, and anti-inflammatory properties of naturally occurring cyclic peptides described here predict their efficacy in the treatment of coronavirus infections including SARS-CoV-2 which causes COVID-19 when administered to patients in need thereof.

One embodiment provides a method of treating an individual for coronavirus infection (such as an alphacoronavirus and/or a betacoronavirus infection) by determining that the individual has or is at risk of developing (e.g., showing early symptoms, history of exposure, etc.) a coronavirus infection and administering an aerosol, injection, and/or infusion that includes a protease inhibitor activity and an anti-inflammatory activity to the individual. The protease inhibitor activity includes one or more of a serine protease inhibitor activity, a cysteine protease inhibitor activity, and a metalloprotease activity, and preferably two or more of these. In some embodiments the protease activity has a serine protease inhibitor activity, a cysteine protease inhibitor activity, and a metalloprotease inhibitor activity. The protease activity can be embodied in a single molecular species, such as a naturally occurring cyclic peptide (e.g., Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16).

One embodiment provides a method for treating SARS-CoV-2 (COVID-19) related pneumonia in a human patient, comprising administering to the patient a composition comprising a naturally occurring cyclic peptide. Another embodiment provides a method for treating SARS-CoV-2 (COVID-19) related pneumonia in a human patient, comprising administering to the patient a composition comprising Peptide 1 (SEQ ID NO. 1) or Peptide 3 (SEQ ID NO. 3). Another embodiment provides a method for treating SARS-CoV-2 (COVID-19) related pneumonia in a hospitalized human patient, comprising intravenously administering to the patient a composition comprising Peptide 1 (SEQ ID NO. 1) or Peptide 3 (SEQ ID NO. 3). Also provided is a method for treating SARS-CoV-2 (COVID-19) related pneumonia in a hospitalized human patient, comprising intravenously administering to the patient a composition comprising an aqueous buffered isotonic solution with a pH of 6±0.1, containing propylene glycol and a final concentration of Peptide 1 (SEQ ID NO. 1) or Peptide 3 (SEQ ID NO. 3) of about 12.5 mg/mL.

One embodiment provides a method of treating and/or preventing a cytokine storm in a patient infected by SARS-CoV-2 or a patient having COVID-19, comprising administering to a patient in need thereof a composition comprising a naturally occurring cyclic peptide.

One embodiment provides a method of reducing the rate of a composite endpoint of death, worsening respiratory insufficiency, or acute insufficiency in renal and/or cardiovascular systems in a patient with COVID-19, comprising administering to a patient in need thereof a composition comprising a naturally occurring cyclic peptide.

One embodiment provides a method of treating or preventing respiratory insufficiency induced by SARS-CoV-2 infection in a patient, comprising administering to a patient in need thereof a composition comprising a naturally occurring cyclic peptide.

In some embodiments the anti-inflammatory activity can be from administering a second protease inhibitor, for example a protease inhibitor activity reduces processing of a pro-inflammatory cytokine. For example, the second protease inhibitor activity can inhibit a sheddase.

In some embodiments the naturally occurring cyclic peptide can be selected to interfere with receptor-based recognition and/or binding of a virus with a host cell of the individual being treated. Such interference can be in the form of competitive or non-competitive binding to a viral protein or to the corresponding host cell membrane receptor.

Alternatively, a naturally occurring cyclic peptide can be selected to reduce expression of a host cell surface receptor for a virus. For example, ACE-2 and neuropilin have been implicated as coreceptors for SARS-CoV-2 infection in humans via interaction with viral spike protein. Accordingly, in certain embodiments, a method is provided for the treatment and/or prevention of SARS-CoV-2 infection, comprising administering to a patient in need thereof, a naturally occurring cyclic peptide that interferes with the interaction between ACE-2 and/or neuropilin with the viral spike protein (for example, by complexing with ACE-2, neuropilin, and/or viral spike protein).

In some embodiments the anti-inflammatory activity can be from administering a second protease inhibitor, for example a protease inhibitor activity reduces processing of a pro-inflammatory cytokine. For example, the second protease inhibitor activity can inhibit a sheddase.

In some embodiments both the protease inhibitor activity and the anti-inflammatory activity are embodied in a single molecular species, such as a naturally occurring cyclic peptide.

In some embodiments, a naturally occurring cyclic peptide is selected or engineered to induce or enhance host expression of host proteins with antiviral activity. Examples of such host antiviral proteins include interferons, expression of which is suppressed by some pathogenic viruses (e.g., SARS-CoV-2). In some embodiments the naturally occurring cyclic peptide is selected or engineered to enhance or increase positive feedback loops within host cells that upregulate expression of such antiviral proteins on exposure to small amounts of the antiviral protein, thereby magnifying the antiviral effect.

Various objects, features, aspects and advantages of the subject matter disclosed herein will become more apparent from the following detailed description of certain embodiments.

### BRIEF DESCRIPTION OF THE DRAWINGS

FIGS. 1A to 1F schematically depict cyclic peptides. FIG. 1A schematically depicts Peptide 1 (SEQ ID NO. 1), Peptide 2 (SEQ ID NO. 2), and Peptide 3 (SEQ ID NO. 3). FIG. 1B schematically depicts Peptide 4 (SEQ ID NO. 4), Peptide 5 (SEQ ID NO. 5), and Peptide 6 (SEQ ID NO. 6). FIG. 1C schematically depicts Peptide 7 (SEQ ID NO. 7), Peptide 8 (SEQ ID NO. 8), and Peptide 9 (SEQ ID NO. 9). FIG. 1D schematically depicts Peptide 10 (SEQ ID NO. 10), Peptide 11 (SEQ ID NO. 11), and Peptide 12 (SEQ ID NO. 12). FIG. 1E schematically depicts Peptide 13 (SEQ ID NO. 13), Peptide 14 (SEQ ID NO. 14), and Peptide 15 (SEQ ID NO. 15). FIG. 1F schematically depicts Peptide 16 (SEQ ID NO. 16).

FIGS. 2A to 2D show typical mean plasma concentration profiles following systemic administration of Peptide 1 (SEQ ID NO. 1). FIG. 2A shows typical results from mice (n=4/time point), FIG. 2B shows typical results from rats (n=6/time point), FIG. 2C shows typical results from cynomolgus monkeys (n=2-12/dosing group) and FIG. 2D shows typical results from vervet (n=1) following single dose IV administration.

FIGS. 3A and 3B show typical mean plasma concentration versus time profiles following a 7-day repeat once daily i.v. administrations of Peptide 1 (SEQ ID NO. 1). FIG. 3A shows typical results in rats at 5 mg/kg/day. FIG. 3B shows typical results in rats at 10 mg/kg/day

FIGS. 4A to 4D show typical mean plasma concentration versus time profiles following repeated i.v. administrations of Peptide 1 (SEQ ID NO. 1) in cynomolgus monkeys. FIG. 4A shows results from administration at 5 mg/kg/day, FIG. 4B shows results from administration at 10 mg/kg/day, (FIG. 4C shows results from administration at 15 mg/kg/day, and FIG. 4D shows results from the recovery group (15 mg/kg, n=4).

FIGS. 5A and 5B show assessments of dose proportionality. FIG. 5A shows Cmax data from cynomolgus monkeys following a single dose administration of Peptide 1 (SEQ ID NO. 1) (0.3, 1, 3, 10, or 15 mg/kg). FIG. 5B shows AUC_{0-∞} data from cynomolgus monkeys following a single dose administration of Peptide 1 (SEQ ID NO. 1) (0.3, 1, 3, 10, or 15 mg/kg).

FIGS. 6A and 6B show typical interspecies allometric correlation. The plot was created based on the results of Peptide 1 (SEQ ID NO. 1) single-dose PK studies. In FIG. 6A the dashed line represents the predicted CL (6.44 L/h) for an adult (70 kg). In FIG. 6B the dashed line represents the predicted Vss (28.0 L) for an adult (70 kg).

FIG. 7 shows typical mean (SD) density of ¹⁴C-Peptide 1 (SEQ ID NO. 1) in tissues and organs 1 h and 24 h after intravenous administration in female rats.

FIG. 8 shows results of naturally occurring cyclic peptides on infection with SARS-CoV-2 or a SARS-CoV-2 pseudovirus treatment in cell culture. FIG. 8A shows effects of Peptide 1 (SEQ ID NO. 1) on infection with a SARS-CoV-2 pseudovirus (SA1) in Vero cells. Vero cells were infected with 160 TCID₅₀ of SA1 virus in DMEM containing 1.5% FBS in 96 well plates, and the indicated concentrations of Peptide 1 (SEQ ID NO. 1) added. After 24 hours of incubation at 37° C., viral titer was quantified by luciferase assay and compared with no SA1 control. FIG. 8B shows results of Peptide 1 (SEQ ID NO. 1), Peptide 2 (SEQ ID NO. 2), and Peptide 5 (SEQ ID NO. 5) on SARS-CoV-2 infections of Calu3 cells. Calu3 2B4 cells were infected with 0.1 MOI SARS-CoV-2 virus in 1% FBS-EMEM in 12 well plates, and the indicated concentrations of Peptide 1 (SEQ ID NO. 1), Peptide 2 (SEQ ID NO. 2), or Peptide 5 (SEQ ID NO. 5) added. After 24 hours of incubation at 37° C., cells were lysed and content of mRNA encoding SARS-CoV-2 Nucleocapsid Phosphoprotein (N) were quantified by RT-PCR.

FIG. 9 shows results of Peptide 1 (SEQ ID NO. 1) treatment on TNF and IL-6 expression in SARS-CoV-2 infected Calu3 cells. Calu3 2B4 cells were seeded at 5×10⁵ cells/well in 12 well plates, infected with SARS-CoV-2 USA-WA1/2020 (at MOI 0.2 (1×10⁵ PFU)) and the indicated concentration of Peptide 1 (SEQ ID NO. 1) or vehicle for 24 hours at 37° C. in 5% CO₂ in DMEM+1% FBS. Medium was removed and mRNA expression analyzed by RT-PCR normalized to ACTB expression.

FIG. 10 shows results of Peptide 1 (SEQ ID NO. 1) and Peptide 3 (SEQ ID NO. 3) on inhibition of TMPRSS2. Caco-2 cells (1×10⁵ per well) were incubated in 96 well plates with 0 to 50 μg/ml of the indicated peptide in the presence of a TMPRSS2 substrate (Boc-Leu-Gly-Arg-AMC) in 50 mM Tris-HCl, pH 7.4. Substrate conversion was monitored in a fluorescence plate reader for 2 hours and inhibition of enzyme activity was expressed relative to a solvent control.

### DETAILED DESCRIPTION

In certain embodiments, compositions are provided that include one or more protease inhibitor activity(ies) and one or more anti-inflammatory activity(ies) and that are effective in treating and/or preventing coronavirus infection. In some embodiments the protease inhibitor activity is directed to more than class of proteases. For example, the protease inhibitor activity can be effective to inhibit serine proteases, cysteine proteases, and/or metalloproteases. The anti-inflammatory activity can be provided by inhibition of enzymes involved in processing or release of pro-inflammatory compounds, such as pro-inflammatory cytokines. In some embodiments multiple protease inhibitor activities can be embodied in a single molecular species. In some embodiments both protease inhibitor activity(ies) and anti-inflammatory activity(ies) can be embodied in a single compound.

In certain embodiments, the compositions provided can be administered by any suitable route, including injection (e.g., subdermal injection, intramuscular injection, intraperitoneal injection), infusion, and inhalation (e.g., of a mist, aerosol, powder, etc.). Compositions that include the protease inhibitor activity(ies) and anti-inflammatory activity(ies) can also include various excipients, which can serve to improve bioavailability, increase solubility, reduce viscosity, etc. In some embodiments, the compositions can be delivered to an individual in need of treatment in any suitable form and by any suitable method, including oral administration and/or topical application to surfaces of the respiratory system. In certain embodiments the compound or compounds, or a composition comprising the same, disclosed herein are delivered to a patient in need of treatment in aerosol form, for example using a nebulizer or inhaler. In some embodiments excipients of the composition can aid in nebulization. In some embodiments, the compositions provided herein are administered intravenously. For example, in some embodiments, the compositions provided herein are administered intravenously once daily for up to 5 consecutive days.

In some embodiments, the numbers expressing quantities of ingredients, properties such as concentration, reaction conditions, and so forth, used to describe and certain embodiments are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments can be approximations, the numerical values set forth in the specific examples are reported as precisely as practicable. The numerical values presented in some embodiments can contain certain errors necessarily resulting from the standard deviation found in their respective testing measurements (e.g., ±1%, ±5%, or ±10%).

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope otherwise claimed. No language in the specification should be construed as indicating any non-claimed element essential to the practice of the disclosure.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Some embodiments utilize ultrastable (e.g., resistant to proteases, extremes of pH, extremes of temperature, etc.), non-immunogenic, nontoxic naturally occurring cyclic naturally occurring cyclic peptides to block or otherwise interfere with infection by betacoronaviruses. Certain betacoronaviruses, including SARS-CoV and SARS-CoV-2, bind to human cells through the binding of the receptor binding domain (RBD) of the viral spike protein to angiotensin converting enzyme 2 (ACE2) expressed on the target host cell. One or more protease is required for proteolytic cleavage of the spike protein, releasing the spike fusion peptide required for viral entry. The naturally occurring cyclic naturally occurring cyclic peptide molecule can have features that block viral infection and proliferation in host tissue. The peptide can have protease inhibition activity against more than one class of protease (e.g., serine proteases, cysteine proteases, metalloproteases, etc.). It should be appreciated that two classes of proteases are involved in the uptake of SARS coronaviruses, i.e., a serine proteinase (TMPRSS2) and a cysteine proteinase Cathepsin B/L (CatB/L). Peptides described herein block both classes of host proteases, providing a method that blocks viral uptake in a manner that exploits the inhibition of host cell protease activity. Notably, the spike protein of SARS-CoV-2 differs from that of SARS-CoV-1 in also being susceptible to cleavage by the serine protease furin, providing an additional mechanism for SARS-CoV-2 entry. Peptides utilized in methods described herein are selected to safely inhibit a broad range of protease activity, and can inhibit both serine protease activity (e.g., TMPRSS2 and/or furin) and cysteine protease (e.g., CatB/L) activity, providing a method that blocks SARS-CoV-2 uptake. Without wishing to be bound by theory, it is believed that this is effective against infection by at least some (or all) viruses (e.g., coronaviruses) that utilize this protease priming pathway for infection.

Compositions and methods described herein exploit host cell machinery by employing naturally occurring cyclic peptides with cross-class antiprotease activities that are active against furin as well as both TMPRSS2 and CatB/L. Moreover, the peptide can be administered either systemically (e.g., intravenously, subcutaneously, intramuscularly) or as an inhaled aerosol (e.g., a gaseous suspension of liquid droplets) which can directly interact with the upper airway surfaces, lower airway surfaces, or both upper and lower airway surfaces.

In some embodiments, a non-immunogenic, nontoxic naturally occurring cyclic naturally occurring cyclic peptides can also interfere with viral binding to the cell surface by interaction with host cell surface receptors and/or viral components that interact with or bind to such host cell surface receptors. For example, a non-immunogenic, nontoxic naturally occurring cyclic peptides can bind to ACE-2 and or neuropilin on a host cell surface in such a way that interaction with the spike protein of SARS-CoV-2 is blocked. Alternatively, a non-immunogenic, nontoxic naturally occurring cyclic peptides can complex with the viral spike protein at a position that blocks interaction with host cell surface receptors. Alternatively, inhibition of protease activity of one or more of furin, TMPRSS2, and CatB/L can interfere with proteolytic processing of coronavirus spike protein necessary to achieve cell entry. Alternatively, inhibition of protease activity within a cell can interfere with processing of coronavirus within the cell.

In some embodiments the naturally occurring cyclic peptide active component can have anti-inflammatory properties that act to reduce pulmonary inflammation, for example by reducing expression and/or processing of pro-inflammatory cytokines. Such inflammation is a fundamental pathophysiologic feature of SARS-CoV pneumonia. The peptide can be selected to be non-toxic to host cells and is non-immunogenic. Such a peptide can also suppress the cytokine storm that accompanies viral pneumonia (such as pneumonia resulting from SARS-CoV-2 infection) by downregulating proinflammatory cytokines, for example by inhibiting processing and/or expression of such proinflammatory cytokines. Proinflammatory cytokines so impacted include TNF, IL-10, IL-6, IL-8, as well as others that are implicated in the pathology associated with ARDS and diffuse alveolar damage. Examples of such peptide include naturally occurring cyclic peptide, such as Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 1 (SEQ ID NO. 15), Peptide 1 (SEQ ID NO. 15), and/or Peptide 16 (SEQ ID NO. 16).

In some embodiments the naturally occurring cyclic peptide active component can enhance expression and/or release of host antiviral proteins, such as interferons when administered to a patient in need thereof. Inhibition of interferon production of such host antiviral proteins has been documented for a number of pathogenic viruses, including coronaviruses. Similarly, a peptide active component can enhance or increase a positive feedback loop in host cells for expression of such antiviral proteins.

One should appreciate that compositions and methods described herein provide readily formulated and easily applied formulations effective in treating coronavirus (e.g., SARS-CoV-2) infection following exposure upon administration of the peptides described herein.

The following discussion provides many example embodiments. Although each embodiment represents a single combination of elements, all possible combinations of the disclosed elements are included. Thus, if one embodiment comprises elements A, B, and C, and a second embodiment comprises elements B and D, then the disclosure is also considered to include other remaining combinations of A, B, C, or D, even if not explicitly recited.

Compositions provided herein include one or more protease inhibitors, which can be selected to inhibit proteases involved in receptor-mediated coronavirus attachment and entry into cells and/or proteases participating in the coronavirus replication process. For example, a protease inhibitor utilized in such compositions can be selected to inhibit a host cell protease (such as furin) that acts to prime a coronavirus (e.g., SARS-CoV-2) spike protein that mediates receptor-based binding to the host cell.

Compositions provided herein can include protease inhibitors that inhibit more than one class of proteases, advantageously providing inhibition at multiple points along the infective process. For example, in some embodiments two or more protease inhibitors can be included in the composition that, in combination, can inhibit serine proteases, cysteine proteases, and/or metalloproteases. In some embodiments a single protease inhibitor (i.e., a single molecular species) is provided that inhibits more than one class of proteases. In one embodiment, a single protease inhibitor is provided in the composition that can inhibit serine proteases, cysteine proteases, and metalloproteases. Use of a single molecular species for this purpose advantageously simplifies both formulation and pharmacokinetics of the composition.

In some embodiments such a species can be non-immunogenic or to have low immunogenicity (i.e., immunogenicity low enough to permit repeated application). In some embodiments a therapeutic species can be selected to be stable (e.g., exhibiting functional stability for at least 24 hours at 37° C.). Reduced immunogenicity and/or stability can be conferred or improved by covalent modification, for example PEGylation.

The mechanism utilized by such a protease inhibitor can be direct or indirect. For example, such a protease inhibitor can act as an analog of a naturally occurring protein substrate. Alternatively, a protease inhibitor can act by reducing expression (e.g., an siRNA) or processing (e.g., release) of a protease. In some embodiments a protease inhibitor can be utilized that has demonstrated inhibitory activity but for which the inhibitory mechanism has not yet been elucidated.

Suitable protease inhibitors include small molecules (e.g., having a molecular weight of less than 1,000 daltons) and polymeric biomolecules (e.g., polynucleic acids, polypeptides, polysaccharides, etc.). Such polymeric biomolecules can be linear, cyclic, or have both linear and cyclic portions. In some embodiments such polymeric biomolecules can include non-naturally occurring monomers (e.g., non-naturally occurring amino acids or nucleic acids). In some embodiments such polymeric molecules can be naturally occurring biomolecules, and can be modified for improved activity, stability, bioavailability, etc.

Suitable small molecule protease inhibitors include, but are not limited to, atazanavir, darunavir, fosamprenavir, indinavir, lopinavir, nelfinavir, ritonavir, saquinavir, tipranavir, amprenavir, DMP450, PNU-140690, ABT-378, PD178390, boceprevir, telaprevir, simeprevir, ombitasvir, paritaprevir, dasabuvir, and ciluprevir. Such inhibitors can be used individually or in any combination.

In some embodiments, the protease inhibitor is a peptide, such as a linear or cyclic peptide. For purposes of this application a peptide can be defined as a linear, cyclic, or mixed linear/cyclic chain of covalently linked amino acids. Such peptides can be naturally occurring protease-inhibiting peptides, such as naturally occurring cyclic peptides.

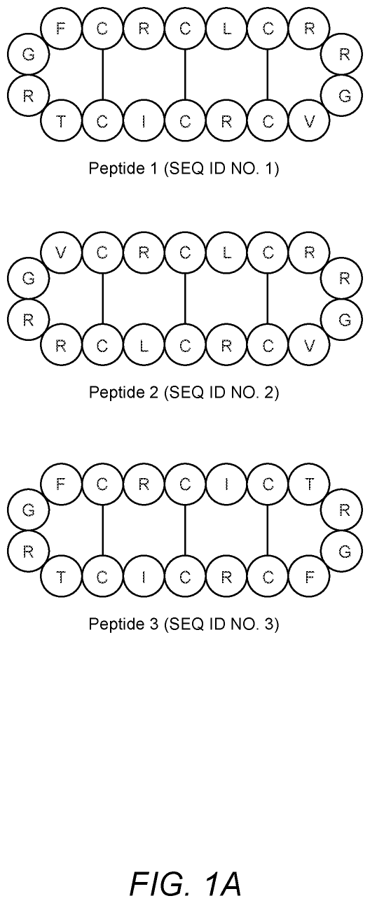

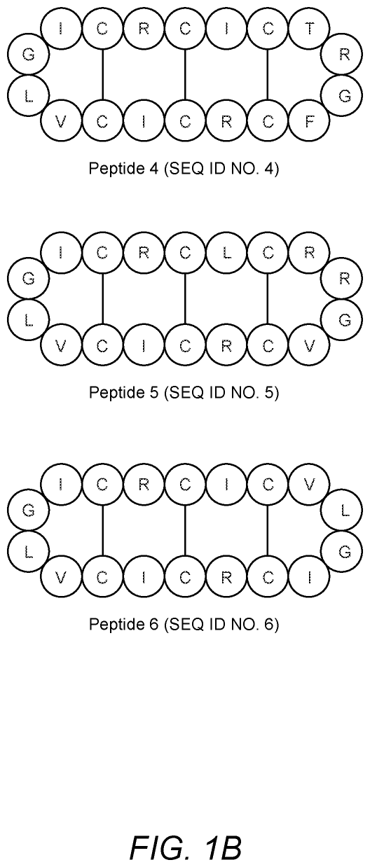

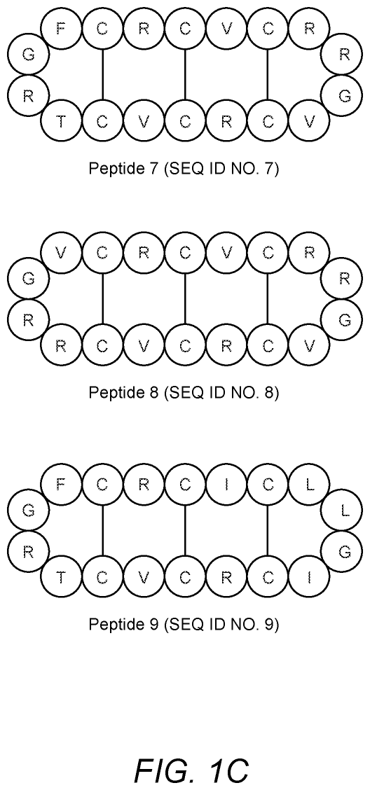

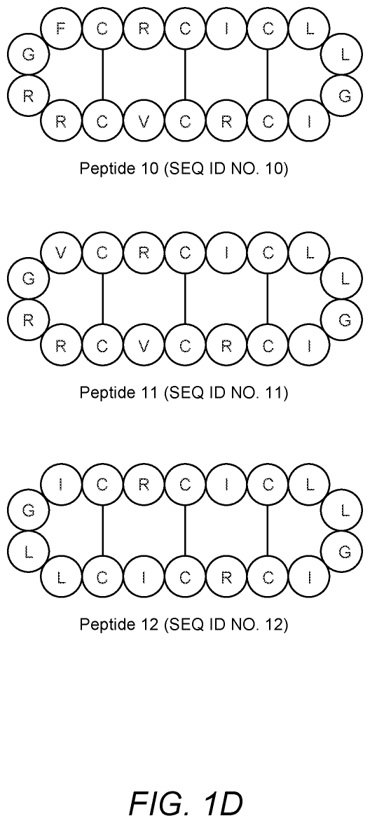

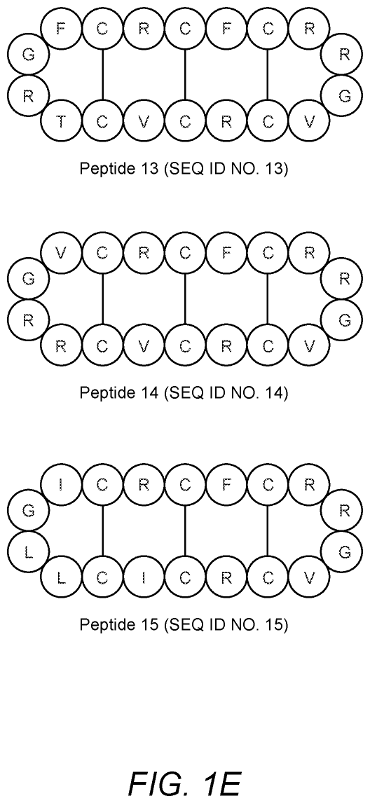

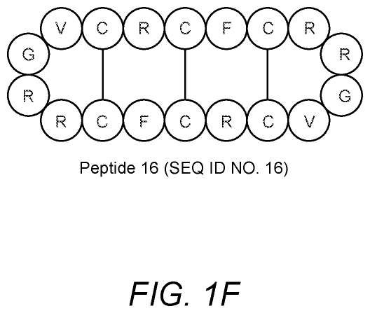

In some embodiments, the protease inhibitor is a cyclic peptide. In some embodiments, the protease inhibitor is a naturally occurring cyclic peptide. Suitable cyclic peptides include Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see, FIGS. 1A to 1F).

Compositions can also include anti-inflammatory compounds. Compositions can provide anti-inflammatory activity, for example by including a compound that provides an anti-inflammatory activity (e.g., inhibition of a protease activity utilized in processing of a pro-inflammatory cytokine). In some embodiments, such a compound can also provide inhibition of serine, cysteine, and metallo-protease activity as described above. Such anti-inflammatory compounds can act directly to inhibit inflammatory processes or pathways of inflammatory processes (e.g., steroids, nonsteroidal COX inhibitors, etc.). In other embodiments the anti-inflammatory compound(s) can act by inhibiting expression (e.g., siRNA) or processing (e.g., release) of proteins that mediate inflammation. In one some embodiments the anti-inflammatory compound(s) can act by inhibiting expression and/or processing of pro-inflammatory cytokines. For example, in some embodiments an anti-inflammatory compound can act by inhibiting the activity of a protease (e.g., a sheddase, TACE, etc.) involved in the processing or release of a pro-inflammatory species (such as a pro-inflammatory cytokine).

Suitable anti-inflammatory species include small molecules (e.g., having a molecular weight of less than 1,000 daltons) and polymeric biomolecules (e.g., polynucleic acids, polypeptides, polysaccharides, etc.). Such polymeric biomolecules can be linear, cyclic, or have both linear and cyclic portions. In some embodiments such polymeric molecules can be naturally occurring biomolecules, and can be modified for improved activity, stability, bioavailability, etc.

In some embodiments, the anti-inflammatory compound is a peptide, such as a linear or cyclic peptide. In some embodiments, such peptides can be naturally occurring anti-inflammatory peptides, such as a naturally occurring cyclic peptide. For example, such an anti-inflammatory peptide can be a theta defensin. Suitable theta defensins include Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see FIGS. 1A-1F).

Embodiments in which a single molecular species is selected that acts as both a protease inhibitor and also as an anti-inflammatory can advantageously simplify both formulation and pharmacokinetics of the formulation.

In some embodiments the single molecular species is a peptide, such as a linear or cyclic peptide. Such peptides can be naturally occurring anti-inflammatory peptides. In some embodiments such a single molecular species can act to both inhibit a protease involved in recognition or binding of a viral pathogen or SARS-CoV-2 (e.g., furin) and also by inhibiting the activity of a protease (e.g., a sheddase, TACE, etc.) involved in the processing or release of a pro-inflammatory species (such as a pro-inflammatory cytokine). For example, such a single molecular species can be a naturally occurring cyclic peptide.

One embodiment provides a method for treating SARS-CoV-2 (COVID-19) related pneumonia in a human patient, comprising administering to the patient a composition comprising a naturally occurring cyclic peptide.

Suitable naturally occurring cyclic peptides include Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see FIGS. 1A-1F).

Another embodiment provides a method for treating SARS-CoV-2 (COVID-19) related pneumonia in a human patient, comprising administering to the patient a composition comprising Peptide 1 (SEQ ID NO. 1) or Peptide 3 (SEQ ID NO. 3). Another embodiment provides a method for treating SARS-CoV-2 (COVID-19) related pneumonia in a hospitalized human patient, comprising intravenously administering to the patient a composition comprising Peptide 1 (SEQ ID NO. 1) or Peptide 3 (SEQ ID NO. 3). Also provided is a method for treating SARS-CoV-2 (COVID-19) related pneumonia in a hospitalized human patient, comprising intravenously administering to the patient a composition comprising an aqueous buffered isotonic solution with a pH of 6±0.1, containing propylene glycol and a final concentration of Peptide 1 (SEQ ID NO. 1) or Peptide 3 (SEQ ID NO. 3) of about 115 mg/mL.

In some embodiments, prior to such treatment or administration, the patient requires intubation or invasive ventilation.

In some embodiments, the method is effective to result in an improvement in any one or more of:
(i) an increase in the percentage of patients discharged from the hospital (e.g., by day 5, 6, 7, 8, 9, 10, 11, 12, 13, or by day 14;
(ii) a decrease in the percentage of patients who require intubation and mechanical ventilation; and
(iii) a decrease in the percentage of patients who require extracorporeal membrane oxygenation; (iv) an increase in the percentage of patients who demonstrate clinical improvement based on validated measures; or
(v) provide a reduction in all-cause mortality.

In some embodiments, such treatment or administration decreases or reduces the use of supplemental oxygen.

In some embodiments, such treatment or administration increases peripheral blood oxygen levels in the patient.

In some embodiments, such treatment or administration decreases the level of C-reactive protein (CRP) in the patient.

In some embodiments, such treatment or administration decreases the level of serum ferritin) in the patient.

In some embodiments, such treatment or administration decreases the level of D-dimer in the patient.

In some embodiments, the patient has a positive RT-PCR assay for SARS CoV-2 in a respiratory tract sample.

In some embodiments, the patient is hospitalized for COVID-19.

In some embodiments, the patient has a radiographic diagnosis of pneumonia.

In some embodiments, the patient suffers respiratory insufficiency.

In some embodiments, the patient does not have a premorbid abnormal pulmonary function or disease.

One embodiment provides a method of treating and/or preventing a cytokine storm in a patient infected by SARS-CoV-2 or a patient having COVID-19, comprising administering to a patient in need thereof a composition comprising a naturally occurring cyclic peptide as described herein (e.g., Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see FIGS. 1A-1F)).

One embodiment provides a method of reducing the rate of a composite endpoint of death, worsening respiratory insufficiency, or acute insufficiency in renal and/or cardiovascular systems in a patient with COVID-19, comprising administering to a patient in need thereof a composition comprising a naturally occurring cyclic peptide as described herein (e.g., Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see FIGS. 1A-1F)).

One embodiment provides a method of treating or preventing respiratory insufficiency induced by SARS-CoV-2 infection in a patient, comprising administering to a patient in need thereof a composition comprising a naturally occurring cyclic peptide as described herein (e.g., Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) (see FIGS. 1A-1F)).

Compositions disclosed herein can be delivered by any suitable route. In some embodiments, a systematic delivery route is utilized. Examples of suitable systematic delivery methods include intravenous, subcutaneous. Intramuscular, and/or intraperitoneal delivery (e.g., injection, infusion, etc.). Inventors have found that formulations of naturally occurring cyclic peptides in conventional aqueous buffers at concentrations that are suitable for administration in effective amounts (e.g., at 5, 15, 20, 25, 30, 35, 40, 45, or 50 mg/mL of higher) can have high viscosity. This high viscosity can complicate both sterile filtration and administration of such formulations via a needle or catheter. Accordingly, formulations suitable for such systemic administration can include compounds that reduce the viscosity of aqueous solutions of naturally occurring cyclic peptides, such as propylene glycol (as described in United States Patent Application Publication No. 2021/0346463).

Compositions described herein can be delivered by any suitable method, including but not limited to injection, topical application, and inhalation. In some embodiments the composition can be administered directly to the pulmonary system of a person in need of treatment for coronavirus infection through application as a liquid droplet or nanodroplet suspension in a gas. Examples of suitable suspensions include aerosols and/or mists of an aqueous liquid solution of a naturally occurring cyclic peptide. In some embodiments such suspensions can be generated using ambient air as a carrier gas. In other embodiments such suspensions can be generated in a carrier gas with a density that is less than air, such as helium, a helium/oxygen mixture, and/or a helium/air mixture.

Liquid droplet suspensions that include a naturally occurring cyclic peptide can be produced by any suitable method. For example, an aqueous solution containing a naturally occurring cyclic peptide can be generated by expressing the aqueous solution through an orifice (such as a nozzle) into the carrier gas. Pressure can be applied through application of a pressurized gas or by application of mechanical pressure to a flexible reservoir of the aqueous solution. In other embodiments a liquid droplet suspension can be generated by sparging a pressurized gas through a reservoir of aqueous solution containing a naturally occurring cyclic peptide. In some embodiments ultrasound can be applied to generate a droplet suspension from an aqueous solution containing a naturally occurring cyclic peptide. In some embodiments a droplet suspension can be generated from an aqueous solution of a naturally occurring cyclic peptide through application of a vibrating mesh to the solution. In some embodiments a naturally occurring cyclic peptide containing liquid droplet suspension can be generated utilizing a nebulizer. In other embodiments a naturally occurring cyclic peptide containing liquid droplet suspension can be generated using a canister that contains a liquid that includes the naturally occurring cyclic peptide and a carrier gas under pressure. Such a canister can be fitted with a pressure valve configured to release a measured dose of liquid and gas when applied to an inhaler or similar metering and/or delivery device equipped with a dispensing nozzle and a mouthpiece. In still other embodiments a mist or vapor of liquid droplets containing a naturally occurring cyclic peptide can be generated by applying a controlled amount of heat to a volume of liquid containing the naturally occurring cyclic peptide and a compound that facilitates formation of a fog, mist, and/or vapor suspension of liquid droplets.

Application of naturally occurring cyclic peptide as droplet suspensions can require the use of high concentrations (e.g. equal to or greater than 1 mg/mL, 2 mg/mL, 3 mg/mL, 4 mg/mL, 5 mg/mL, 10 mg/mL, 15 mg/mL, or 20 mg/mL) of these peptides in aqueous solution, due to the relatively small liquid volume delivered. Aqueous solutions of naturally occurring cyclic peptides, however, become highly viscous as the concentration of the peptide increases. This high viscosity can interfere with the generation of a gaseous suspension of liquid droplets.

The effects of viscosity on droplet generation are complex, and dependent to some extent on the technology used to generate the droplets. For example, ultrasonic methods for generating microdroplets are ineffective when viscosity of the solution is too high. Generally speaking, however, lower viscosity solutions provide less resistance to droplet generation and can result in the production of smaller mean droplet diameter and increased production. Such small droplets (e.g., less than about 200 micron, 100 micron, 50 micron, 25 micron, or 10 micron in mean diameter) can provide efficient delivery deep into the lungs on inhalation, however the requirements for efficient production of such small droplets is incompatible with the need to deliver a defensin peptide at high concentrations.

It has been found that inclusion of low molecular weight polyols (such as propylene glycol, etc.) at concentrations in excess of about 0.5% (v/v) can dramatically decrease the viscosity of aqueous solutions of t θ-defensins. For example, suitable solutions for generation of liquid microdroplet suspensions in a carrier gas can have a viscosity of less than 600 cP, 500 cP, 400 cP, 300 cP, 250 cP, 200 cP 150 cP, or 100 cP. In one some embodiments a liquid droplet suspension in a carrier gas is generated from a solution of a naturally occurring cyclic peptide in an aqueous buffer that includes propylene glycol. In such an embodiment propylene glycol can be present at from 0.1%, 0.25%, 0.5%, 1%, 1.5%, 2%, 3%, 4%, 5%, or more than 5% (v/v), where the concentration of propylene glycol is selected to provide a desired liquid droplet mean diameter and/or production efficiency suitable for delivery by the chosen production method.

It should be appreciated that low molecular weight polyol compounds (e.g., propylene glycol, ethylene glycol, etc.) can, advantageously, support the formation of consumable mists, fogs, and/or vapors of liquids containing pharmaceutically active compounds using inhalation devices that include a simple heating element. Examples of such devices include e-cigarettes, vaping devices, and similar devices. Formulations for use in such devices can include stabilizers that improve or preserve activity and/or heat stability of protease inhibitors and/or anti-inflammatory compounds of compositions provided herein.

In certain embodiments a naturally occurring cyclic peptide can be provided by injection, such as a subcutaneous injection, an intramuscular injection, and/or an intravenous infusion. Certain injection embodiments include an intravenous infusion. Such an approach can be advantageously be used to provide high bioavailability of the peptide, particularly in acute patients with compromised pulmonary function. In such embodiments a naturally occurring cyclic peptide (such as Peptide 1) can be provided as a concentrated (e.g., greater than 2.5 mg/mL, 5 mg/mL, 7.5 mg/mL, 10 mg/mL, 12.5 mg/mL, 15 mg/mL, 17.5 mg/mL, 20 mg/mL, or greater) solution in a pharmaceutically acceptable aqueous buffer suitable for intravenous infusion. As noted above, concentrated solutions of naturally occurring cyclic peptide can be highly viscous. Accordingly, in certain embodiments the pharmaceutically acceptable aqueous buffer can include propylene glycol at a concentration of about 0.1% v/v, 0.25% v/v, 0.5% v/v, 0.75% v/v, 1% v/v, 1.5% v/v, 2% v/v or greater) to reduce viscosity and simplify administration.

Given the acute nature of SARS-CoV-2 pneumonia, it is contemplated that administration of relatively high doses of the peptide (e.g., 1 mg/kg, 2 mg/kg, 5 mg/kg, 7 mg/kg, 10 mg/kg, 12 mg/kg, 15 mg/kg, or more) can be included in treatment protocols. Some embodiments of the methods provided herein, such as for treating a patient suffering from COVID-19, include administering to a human in need thereof (i.e., having or at risk of infection by SARS-CoV-2), a pharmaceutical composition that includes a naturally occurring cyclic peptide at a concentration of up to 50 mg/mL in an aqueous solution that includes about 0.5% to about 1.5% v/v propylene glycol. Such a pharmaceutical composition can have a pH of from about 5.0 to about 7.0. In certain embodiments, can be effective when administered systemically, for example intravenously.

The safety profile of naturally occurring cyclic peptides (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)) indicates that a wide range of dosing schedules can be used. For example, naturally occurring cyclic peptides can be administered (e.g., systemically and/or by inhalation) once an hour, every 2 hours, every 4 hours, every 6 hours, every 8 hours, every 12 hours, once daily, every two days, three times a week, twice a week, weekly, every two weeks, once a month, or any suitable interval. In some embodiments the dosing schedule (in terms of either or both of mg/kg naturally occurring cyclic peptide provided and frequency of provision) can be adjusted during the course of treatment. Such adjustment can be made on the basis of symptoms, viral titer, and/or historical data.

In some embodiments two or more naturally occurring cyclic peptides can be administered simultaneously. In such embodiments the naturally occurring cyclic peptides used can be provided in any suitable ratio. For example, a mixture of Peptide 1 (SEQ ID NO. 1) and Peptide 2 (SEQ ID NO. 2) can be administered in a Peptide 1:Peptide 2 ratio ranging from 100:1 to 1:100, or any intermediate value. In such embodiments the selection of naturally occurring cyclic peptides and/or the ratio between the naturally occurring cyclic peptides can be adjusted during the course of treatment. Such adjustment can be made on the basis of symptoms, viral titer, and/or historical data.

Some embodiments of the methods provided herein, such as for treating a patient suffering from COVID-19, can include administering to a human in need thereof, a pharmaceutical composition that includes a naturally occurring cyclic peptide at a concentration of about 10 to about 50 mg/mL in an aqueous solution that includes about 0.5% to about 1.5% v/v propylene glycol. Such a pharmaceutical composition has a pH of from 5.0 to 7.0.

Similarly, some embodiments of the methods provided herein, such as for treating a patient suffering from COVID-19, include administering to a human in need thereof, a pharmaceutical composition that includes a naturally occurring cyclic peptide at a concentration of about 10, about 30 or about 50 mg/mL in an aqueous solution that includes about 0.5% to about 1.5% v/v propylene glycol. Such a pharmaceutical composition can have a pH of from about 5.0 to about 7.0.

Some embodiments of the methods provided herein, such as for treating a patient suffering from COVID-19, include administering to a human in need thereof, a pharmaceutical composition comprising a naturally occurring cyclic peptide at a concentration of about 10, about 30 or about 50 mg/mL in an aqueous solution that includes about 1% v/v propylene glycol. Such a pharmaceutical composition cab gave a pH of about 6.

Solutions of naturally occurring cyclic peptides can be administered by injection (e.g., subdermal injection, intramuscular injection, intravenous injection, intraperitoneal injection) and/or infusion at doses ranging from 0.1 mg/kg to 15 mg/kg, preferably at doses ranging from about to 0.1 mg/kg to about 0.3 mg/kg. Intravenous administration of a naturally occurring cyclic peptide solution can be provided as a single daily infusion, as a series of infusions through the day, or as a continuous infusion throughout the course of treatment. Suitable courses of treatment range from one day to up to 30 days or more. In some embodiments a solution of naturally occurring cyclic peptide can be administered once a day for a period of from 3 to 7 days.

In some embodiments compositions and methods as described above can be implemented as part of co-therapy with one or more therapies to enhance or improve response of the innate immune system to viral infection. Examples include co-therapy with azithromycin, CSY0073, and Mac26.

In certain embodiments, provided is a method for treating or ameliorating one or more symptoms of COVID-19 in a human, comprising administering to a human patient in need thereof, an effective amount of a composition comprising a naturally occurring cyclic peptide. In certain embodiments, the naturally occurring cyclic peptides Peptide 1 (SEQ ID NO. 1). In certain embodiments, the naturally occurring cyclic peptide is Peptide 3 (SEQ ID NO. 3). In certain embodiments, the administering comprises intravenous administration. In certain embodiments, the administering comprises aerosol administration.

In certain embodiments, provided is a method for treating or ameliorating one or symptoms of a SARS-CoV-2 infection in a human, comprising administering to a human patient in need thereof, an effective amount of a composition comprising a naturally occurring cyclic peptide (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)). In certain embodiments, the naturally occurring cyclic peptide is Peptide 1 (SEQ ID NO. 1). In certain embodiments, the naturally occurring cyclic peptide is Peptide 3 (SEQ ID NO. 3). In certain embodiments, the administering comprises intravenous administration. In certain embodiments, the administering comprises aerosol administration.

In certain embodiments, provided is a method preventing or lessoning one or symptoms of a SARS-CoV-2 infection in a human suspected of having a SARS-CoV-2 infection or a human who has come into close contact with a person diagnosed (or suspected of having) a SARS-CoV-2 infection, comprising administering to a human patient in need thereof, an effective amount of a composition comprising a naturally occurring cyclic peptide (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)). In certain embodiments, the naturally occurring cyclic peptide is Peptide 1 or Peptide 3. In certain embodiments, the naturally occurring cyclic peptide is Peptide 1 (SEQ ID NO. 1) or Peptide 3 (SEQ ID NO. 3). In certain embodiments, the naturally occurring cyclic peptide is Peptide 1 (SEQ ID NO. 1). In certain embodiments, the administering comprises intravenous administration. In certain embodiments, the administering comprises aerosol administration.

In certain embodiments, provided is a method for treating one or more symptoms associated with a SARS-CoV-2 infection, comprising administering to an individual in need thereof a composition comprising a naturally occurring cyclic peptide (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)). In certain embodiments, the naturally occurring cyclic peptide is provided at a dose of from 0.1 mg/kg to 15 mg/kg. In certain embodiments, the composition is delivered intravenously. In certain embodiments, the composition is administered once daily for three or more days. In certain embodiments, the composition further comprises propylene glycol. In certain embodiments, composition is administered as an aerosol. In certain embodiments, the one or more symptoms is selected from the group consisting of lung inflammation, respiratory insufficiency and respiratory distress.

In certain embodiments, provided is a method for treating one or more symptoms associated with a SARS-CoV-2 infection, comprising administering to an individual in need thereof a composition comprising Peptide 1 (SEQ ID NO. 1). In certain embodiments, Peptide 1 (SEQ ID NO. 1) is administered at a dose of from 0.1 mg/kg to 15 mg/kg. In certain embodiments, the composition is delivered intravenously. In certain embodiments, the composition is administered once daily for three or more days. In certain embodiments, the composition further comprises propylene glycol. In certain embodiments, composition is administered as an aerosol. In certain embodiments, the one or more symptoms is selected from the group consisting of lung inflammation, respiratory insufficiency and respiratory distress.

In certain embodiments, provided is a method of treating an individual for SARS-CoV-2 infection, comprising determining that the individual has a SARS-CoV-2 infection; and administering an aerosol to the individual, where the aerosol comprises a first protease inhibitor activity and an anti-inflammatory activity (e.g., in the form of one or more naturally occurring cyclic peptide). In certain embodiments, the aerosol comprises propylene glycol. In certain embodiments, the naturally occurring cyclic peptide is provided at a dose of from 0.1 mg/kg to 15 mg/kg. In certain embodiments, the first protease inhibitor activity and an anti-inflammatory activity is Peptide 1 (SEQ ID NO. 1). In certain embodiments, the first protease inhibitor activity and an anti-inflammatory activity is Peptide 3 (SEQ ID NO. 3).

In certain embodiments, provided is a method of preventing development of a SARS-CoV-2 infection in an individual, comprising determining that the individual is at risk of developing a coronavirus infection; and administering an aerosol to the individual, wherein the aerosol comprises a first protease inhibitor activity and an anti-inflammatory activity (e.g., in the form of a naturally occurring cyclic peptide). In certain embodiments, the aerosol comprises propylene glycol. In certain embodiments, the naturally occurring cyclic peptide is provided at a dose of from 0.1 mg/kg to 15 mg/kg. In certain embodiments, the first protease inhibitor activity and an anti-inflammatory activity is Peptide 1 (SEQ ID NO. 1). In certain embodiments, the first protease inhibitor activity and an anti-inflammatory activity is Peptide 3 (SEQ ID NO. 3).

In certain embodiments, the first protease inhibitor activity comprises at least one of a serine protease inhibitor activity, a cysteine protease inhibitor activity, and/or a metalloprotease activity.

In certain embodiments, the first protease inhibitor activity is embodied in a single molecular species, for example a naturally occurring cyclic peptide.

In certain embodiments, the anti-inflammatory activity comprises a second protease inhibitor activity. In certain embodiments, the second protease inhibitor activity reduces processing of a pro-inflammatory cytokine. In certain embodiments, the second protease inhibitor activity inhibits a sheddase.

In certain embodiments, the first protease inhibitor activity and the anti-inflammatory activity are embodied in a single molecular species. In certain embodiments, the single molecular species is a naturally occurring cyclic peptide.

In certain embodiments, the naturally occurring cyclic peptide comprises 18 amino acids and three disulfide bonds.

In certain embodiments, the individual has an underlying medical condition.

In certain embodiments, the individual is asymptomatic.

In certain embodiments, the individual has been diagnosed with viral pneumonia.

In certain embodiments, the individual is hospitalized.

Another embodiment provides a method for treating SARS-CoV-2 (COVID-19) related pneumonia in a hospitalized human patient, comprising intravenously administering to the patient a composition comprising a naturally occurring cyclic peptide (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)).

Another embodiment provides a method for treating SARS-CoV-2 (COVID-19) related pneumonia in a hospitalized human patient, comprising intravenously administering to the patient a composition comprising Peptide 1 (SEQ ID NO. 1).

Also provided is a method for treating SARS-CoV-2 (COVID-19) related pneumonia in a hospitalized human patient, comprising intravenously administering to the patient a composition comprising an aqueous buffered isotonic solution with a pH of 6±0.1, containing propylene glycol and a final concentration of Peptide 1 (SEQ ID NO. 1) of about 12.5 mg/mL.

In certain embodiments, the individual is receiving pharmacologic thromboprophylaxis.

In certain embodiments, the individual has not been diagnosed or does not suffer from premorbid abnormal pulmonary function or disease.

In certain embodiments, the individual is not concurrently receiving systemic corticosteroids or other immunomodulators or immunosuppressant drugs.

In certain embodiments, provided is a method for preventing or decreasing the need for invasive mechanical ventilation in a patient population suffering from COVID-19 or a SARS-CoV-2 infection, comprising administering to a human patient in need thereof, an effective amount of a composition comprising a naturally occurring cyclic peptide (e.g., Peptide 1 (SEQ ID NO. 1), Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)). In certain embodiments, the theta defensin is Peptide 1 (SEQ ID NO. 1). In certain embodiments, the administering comprises intravenous administration.

In certain embodiments, compositions and methods as described above can be implemented as part of cotherapy with conventional anti-viral therapy. For example, treatment with naturally occurring cyclic peptides can be carried out in combination with antibodies directed to portions of the SARS-CoV-2 virus. Such antibodies can be polyclonal (e.g., immunoglobulins of convalescent serum) or monoclonal. Examples of suitable monoclonal antibodies include bamlanivimab, etesevimab, casirivimaband, and/or sotrovimab. Cyclic peptides can also be used in combination with small molecule (i.e., molecular weight of less than 1 kD) drugs directed to SARS-CoV-2. Examples of suitable small molecule drugs include remdesivir, Paxlovid (nirmatrelvir/ritonavir), or molnupiravir.

In certain embodiments, the method further comprises administration of one or more additional therapeutic agent(s) for treating COVID-19. In such embodiments a synergistic (i.e., greater than additive effect) can be observed with such combined therapy.

In certain embodiments, the additional therapeutic agent is convalescent plasma, a therapeutic vaccine, a corticosteroid (e.g., dexamethasone, hydrocortisone, or methylprednisolone), baricitinib, a monoclonal antibody (MAB) (e.g., bamlanivimab (LY-CoV555), casirivimab and imdevimab (REGN-COV2), etc.), and/or remdesivir.

In certain embodiments, the additional therapeutic agent is hydroxychloroquine, chloroquine, azithromycin, an IL-6 inhibitor (e.g., tocilizumab or sarilumab), kinase inhibitor (e.g., Acalabrutinib (Calquence), Baricitinib (Olumiant), Ruxolitinib (Jakafi), Tofacitinib (Xeljanz), etc.), interferon-alfa (IFN-α), interferon-beta (IFN-β), Kaletra (lopinavir/ritonavir), ivermectin, Tamiflu (oseltamivir), favipiravir, umifenovir, galidesivir, colchicine, convalescent plasma, a corticosteroid (e.g., dexamethasone, hydrocortisone, or methylprednisolone), baricitinib, a monoclonal antibody (MAB) (e.g., bamlanivimab (LY-CoV555) casirivimab and imdevimab (REGN-COV2), etc.), remdesivir, Paxlovid (nirmatrelvir/ritonavir), or molnupiravir.

It is contemplated that a suitable naturally occurring cyclic peptide, such as Peptide 1 (SEQ ID NO. 1) will effectively treat patients infected with SARS-CoV-2 suffering from COVID-19 (e.g., lung inflammation and respiratory distress) irrespective of mechanism, or reduce the probability of infection of a person at risk of SARS-CoV-2 infection (symptomatic or asymptomatic) due to exposure and/or underlying medical condition.

### Example 1: Escalating Dose Study of Peptide 1 (SEQ ID NO. 1), for Treatment of Hospitalized Human Patients with SARS-CoV-2 (COVID-19) Related Pneumonia

The following protocol is intended to assess the effect of Peptide 1 (SEQ ID NO. 1) on the rate of COVID-19 disease progression including the need for invasive mechanical ventilation, as well as the safety, tolerability, and immunogenicity of intravenous administration of Peptide 1. This study evaluates Peptide 1 by intravenous (IV) administration as a therapy for COVID-19.

The IV solution will be composed of an aqueous buffered isotonic solution with a pH of 6±0.1, containing propylene glycol and a final concentration of Peptide 1 (SEQ ID NO. 1), of 12.5 mg/mL. Peptide 1 (SEQ ID NO. 1) is a naturally occurring cyclic, cationic, tri-disulfide peptide composed of 18 amino acids with a molecular weight of 2081.63 Da.

### Study Design

Randomized, blinded, vehicle-controlled dose-escalation study. Initial enrollment and treatment will be conducted as an inpatient study. Patients will be randomized 2:1, Peptide 1 treatment versus vehicle control.

Patients (18 total) will be sequentially assigned to 1 of 3 cohorts (Peptide 1 (SEQ ID NO. 1) dose levels of 0.10 mg/kg, 0.30 mg/kg, and 0.50 mg/kg) with 6 randomized patients per cohort 2:1 (4 with Peptide 1 (SEQ ID NO. 1): 2 with vehicle control). The standard of care currently implemented at the study site(s) for COVID-19 patients will be used in each arm of the study and documented in each patient's case report form (CRF).

After enrollment and randomization, patients will be treated with study drug (Peptide 1 (SEQ ID NO. 1) or vehicle control) by IV administration based on their assigned dose cohort (Cohort 1=0.10 mg/kg [low dose], Cohort 2=0.30 mg/kg [mid dose], and Cohort 3=0.50 mg/kg [high dose]). Patients in Cohort 1 will be infused with 0.10 mg/kg of Peptide 1 (SEQ ID NO. 1) or vehicle once daily for 5 consecutive days followed by a pause for safety review by the data safety monitoring board (DSMB).

Patients in Cohort 2 will be infused with 0.30 mg/kg of Peptide 1 (SEQ ID NO. 1) or vehicle once daily for 5 consecutive days followed by a pause for safety evaluation by the DSMB. Patients in Cohort 3 will be infused with 0.50 mg/kg Peptide 1 or vehicle once daily for 5 consecutive days followed by evaluation by the DSMB. Vehicle control will be infused at the same rate as Peptide 1 (SEQ ID NO. 1) (active) drug.

Study drug infusions and associated assessments will be conducted as inpatient procedures. Patients will be discontinued from treatment if they progress to a potentially life-threatening adverse event or severe toxic reaction, require mechanical ventilation (intubation), or experience drug-related cytopenia.

Follow up safety evaluations will be conducted for all patients after the last infusion, and patients will be followed until all SAEs and AEs assessed as possibly related have resolved or stabilized. Patients discharged from the hospital will also be followed remotely by telephone (or telehealth, if possible), and outpatient visits will be scheduled at 14, 21, 35, and 65 days after enrollment.

Patient Population: Adult patients (≥18 years old) hospitalized with laboratory confirmed SARS-CoV-2 infection who have viral pneumonia. Patients eligible for this study do not require intubation for mechanical ventilation.

The dose for each patient will be 0.10 mg/kg, 0.30 mg/kg, or 0.50 mg/kg of study drug or equivalent volume of vehicle, given intravenously once daily for 5 days at 1 mL/min (100-minute infusion duration). An infusion volume of 100 mL will be used.

The study drug will be provided in 2.0 mL single use, clear, borosilicate glass vials with a rubber septum and aluminum crimp. Each vial contains 1.0 mL of either Peptide 1 or vehicle control. The final concentration of Peptide 1 is 12.5 mg/mL. The study drug will be stored at 2-8° C. in the site pharmacy until the time of dose preparation.

The study will be a minimum of 65 days duration for each participant. In certain cases, patients may receive corticosteroids for COVID-19 per site standard of care. Drugs authorized under emergency authorization for COVID-19 treatment (e.g., remdesivir) may also be used as concomitant medications.

Blood samples for pharmacokinetic assessment will be collected pre-infusion (within 15 minutes of initiating the infusion) and post-infusion at 0.5, 2, 6, 12, and 24 hours on the days of the first and last infusion, as well as a single assessment on dosing Day 2-4 (either pre-infusion or between 1-10 hours post-infusion) and at follow up Days 14, 21, 35, and 65. On Day 2, the PK blood sample must be collected pre-infusion and will serve as the Day 1 24-hour timepoint. The date and time of each PK sample will be recorded.

PK samples should be collected from the arm contralateral to the arm used for study drug infusion. Plasma samples will be stored at −20° C. until analyzed. Samples will be analyzed using a validated method for the detection of Peptide 1 in human plasma. Blood sample volumes collected will be 2.0 mL per time point, which will be divided into 2 samples prior to shipment to the PK laboratory.

Safety endpoints to be evaluated include incidence and severity of adverse events, clinical exams, labs, and vital signs and assessment of immunogenicity following IV infusion.

Clinical outcome endpoints to be evaluated include proportion of patients requiring intubation and invasive ventilation at each visit, percentage of days requiring supplemental oxygen (overall, while in hospital and while at home), and overall survival (OS), defined as the time from enrollment until death from any cause.

PK will be assessed based on pre-infusion and post-infusion blood samples obtained for the first and last infusion and additional sparse sampling during the treatment and follow-up periods.

Clinical and laboratory outcome measures include peripheral blood oxygen levels (SpO2 over time will be measured by pulse oximetry), inflammatory biomarkers: TNF-α, IL-6, C-reactive protein, serum ferritin, and D-dimer.

Statistical Methods: The safety population is defined as all enrolled patients who receive at least one dose of study treatment. Continuous data will be summarized using descriptive statistics (number, mean, median, standard deviation, minimum, and maximum values). Categorical data will be summarized using frequency tables (frequencies and percentages).

Serum concentration of Peptide 1 will be measured following IV infusion and during follow-up.

### Example 2: In Vivo Safety Studies

The following studies have been performed to confirm safety of systemic administration of cyclic peptides such as Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16).

### Summary

Single and multiple-dose studies performed in rats and cynomolgus monkeys demonstrated the excellent safety profile of intravenous Peptide 1 (SEQ ID NO. 1) administration. Repeat administration of Peptide 1 (SEQ ID NO. 1) was well tolerated in rats at doses up to 10 mg/kg, and therefore, the NOAEL in rats was established at 10 mg/kg/day. Treatment-related mortality and adverse clinical signs were observed in rats treated at 20 mg/kg, including cold to touch, abnormal body color, inability to walk, extreme dehydration, and tremors. In cynomolgus monkeys, single and repeated daily dose administration of Peptide 1 (SEQ ID NO. 1) was tolerated up to 15 mg/kg/day, with no major treatment-related adverse findings or toxicities. Given the lack of adverse findings, the NOAEL was established at 15 mg/kg/day in cynomolgus monkeys. The NOAEL in the cynomolgus monkeys was established at a higher dose when compared with the rats, demonstrating that Peptide 1 (SEQ ID NO. 1) was better tolerated in cynomolgus monkeys. Most changes noted in hematological, serum chemistry, and coagulation parameters in both rats and cynomolgus monkeys were determined as non-adverse due to their low magnitude, lack of consistency between the two species, lack of dose dependence, and/or reversibility by the end of the recovery period. Therefore, these data demonstrate the safety of intravenous Peptide 1 (SEQ ID NO. 1) at a dose up to 10 mg/kg/day in rats and 15 mg/kg/day in cynomolgus monkeys.

The PK of intravenous Peptide 1 (SEQ ID NO. 1) following single and multiple ascending doses in multiple species is characterized by extensive tissue distribution and prolonged elimination. The volume of distribution (Vss) normalized to a bodyweight of the animals receiving 5 mg/kg of Peptide 1 (SEQ ID NO. 1) varied across species, with 1,048, 1,461, 550 mL/kg, in mice, rats, and cynomolgus monkeys, respectively. The relatively large Vss indicates that Peptide 1 (SEQ ID NO. 1) extensively distributes to tissues. The biodistribution study confirmed extensive tissue distribution, particularly in the liver. The prolonged elimination half-life observed in cynomolgus monkeys in the recovery group (47.2 h) is suggestive of tissue redistribution.

Analysis of the single- and multiple ascending dose studies in rats and monkeys showed a greater than dose-proportional increase in AUC_{0-∞} and Cmax suggestive of nonlinear PK. Several therapeutic proteins exhibit nonlinear PK mediated by different mechanisms. For instance, exenatide and recombinant human interferon (IFN) (e.g., IFN-β1a) display nonlinear kinetics due to the saturation of the elimination pathway, such as target-mediated drug disposition (TMDD). Alternatively, nonlinear PK of cyclosporin A and erythropoietin were attributable to the saturation of tissue binding and receptors in target tissues, respectively. The widespread distribution of ¹⁴C-Peptide 1 (SEQ ID NO. 1) in rats in the biodistribution study, which could explain the large Vss estimated in the preclinical PK studies, suggests that saturation of the peptide within these tissues may be one potential source of nonlinearity. Since the greatest accumulation of Peptide 1 (SEQ ID NO. 1) occurred in the liver and kidney, a dose-dependent decrease in CL observed in rats and cynomolgus monkeys could also explain the nonlinearity. Consistent with data from other small peptides (<10 kDa) which are predominately cleared through the glomerular filtration, appreciable amounts of ¹⁴C-Peptide 1 (SEQ ID NO. 1) were recovered from the urine (accounting for 7% at 24 h). In addition, a relatively significant portion was recovered in feces (accounting for 4% at 24 h) indicating that elimination of Peptide 1 (SEQ ID NO. 1) occurs through renal and biliary excretion. Therefore, the nonlinear PK of Peptide 1 (SEQ ID NO. 1) may be attributable to saturation of uptake and/or efflux transporters present in the liver or kidney. However, the definitive role of hepatic/renal transporters in the distribution and elimination of Peptide 1 (SEQ ID NO. 1) might be elucidated with further investigation.

Interspecies allometric scaling provides methods for extrapolating PK data from preclinical species to humans and is commonly used to predict an appropriate dosage for FIH clinical trials. Since Peptide 1 (SEQ ID NO. 1) is believed to follow linear PK at the HED for efficacy, as evidenced by dose-proportional increases in the AUC_{0-∞} at lower doses in cynomolgus monkeys (0.3-3 mg/kg), interspecies allometric scaling using simple allometry was performed to predict human PK. For macromolecules that are renally excreted, human CL can be adequately predicted using the simple allometric equation. As three or more preclinical species are typically needed to reliably scale the parameters to humans, available single-dose data from mice and vervet were included in the analysis. The estimated allometric scaling exponents of 0.829 and 0.866 for CL and Vss respectively, agree with the values reported for other therapeutic proteins, which are 0.65-0.84 for CL and 0.84-1.02 for Vss. The target AUC was previously established in a mouse model of LPS-induced ALI, where a single subcutaneous injection of 5- or 25 mg/kg Peptide 1 (SEQ ID NO. 1), which resulted in mean AUC_{0-∞} of 3,869 and 9,001 ng*h/mL respectively, led to a significant decrease in airway neutrophil burden and inflammatory cytokines/chemokines without mortality. The HED was determined using the predicted human CL (6.44 L/h) from interspecies allometric scaling and the target AUC for efficacy (from the murine model of endotoxin-induced ALI). The HED equivalent to NOAEL in cynomolgus monkeys of 15.9 mg/kg was higher than both the HED equivalent to NOAEL and LOAEL in rats (3.1 and 11.7 mg/kg, respectively). Since cynomolgus monkeys are more physiologically similar to humans than rats, doses up to 15.9 mg/kg may be tolerated in humans.

### Pharmacokinetics

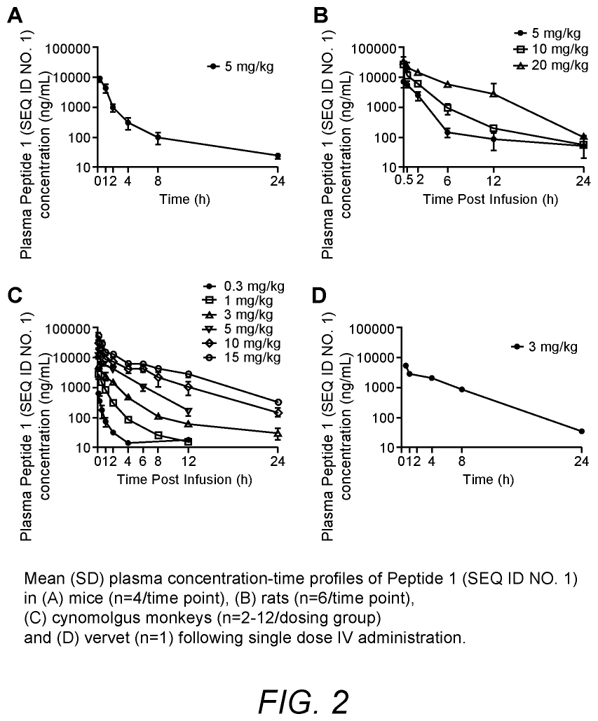

Mouse studies: The mean plasma concentration-time profile after single-dose administration of Peptide 1 (SEQ ID NO. 1) in mice is shown in FIG. 2A, and the corresponding pharmacokinetic (PK) parameters calculated by non-compartmental analysis (NCA) are listed in Table 1. Due to terminal blood collection from each mouse, a pooled PK result was generated. After single i.v. bolus administration, Peptide 1 (SEQ ID NO. 1) displayed a biphasic profile, with a relatively short distribution phase followed by a longer elimination phase with a half-life of 6.05 hours.

TABLE 1

| Parameters | Estimate (SE) |
|---|---|
| Cₘₐₓ (ng/mL) | 8,919 (1,088) |
| λz (h⁻¹) | 0.114 |
| AUCτ (ng*h/mL) | 12,282 |
| AUC_{0-∞} (ng*h/mL) | 12,497 |
| CL (mL/h/kg) | 400 |
| MRT (h) | 2.62 |
| Vss (mL/kg) | 1,048 |

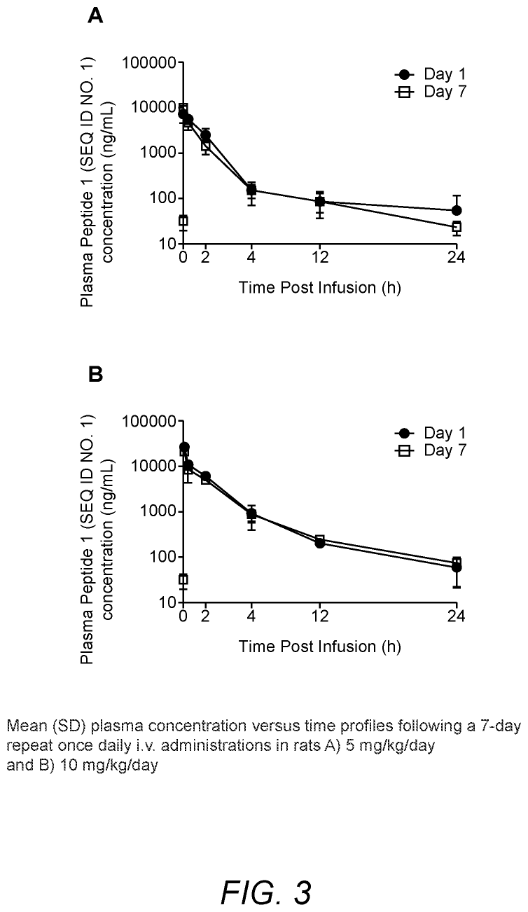

Rat studies: The mean plasma concentration-time profiles in rats following single (5, 10, or 20 mg/kg) or multiple doses (5 or 10 mg/kg/day) administrations of Peptide 1 (SEQ ID NO. 1) are depicted in FIG. 2B and FIGS. 3A/3B, respectively, and the corresponding PK parameters are summarized in Table 2. Of the total 180 infusions from 36 rats, five infusions deviated more than 10% from the intended 20-min infusion. However, these deviations did not occur on blood PK sampling days and therefore did not influence the PK analysis. Due to sparsely sampled data per rat, a pooled PK result was generated in WinNonlin. Plasma levels of Peptide 1 (SEQ ID NO. 1) were undetectable during the recovery period (Day 25) in all rats, except for one female rat that received 10 mg/kg, which had a concentration of 12.5 ng/mL. Early removal of the rats in the 20 mg/kg group precluded the PK analysis with repeat dosing at this dose level. The Cmax was slightly higher in females compared to male rats, but the differences did not reach statistical significance. While both Cmax and AUC_{0-∞} appeared to increase proportionally to the dose based on 95% confidence interval (CI) of the slope including 1, (Cmax, Y=1.110*X+8.936 [Slope 95% CI: 0.7066 to 1.513], r²=0.6803; AUC0-∞, Y=1.543*X+9.448 [Slope 95% CI: 0.4460 to 2.639], r²=0.9969), a comprehensive analysis of dose proportionality in rats was limited due to pooled calculations of AUC_{0-∞} at each dose level, and the relatively narrow range of doses tested. The AUCτ on Day 7 was slightly lower (22% and 19% for the 5- and 10 mg/kg groups, respectively) when compared with their AUC_{0-∞} on Day 1, indicating no significant drug accumulation.

TABLE 2
Single and multiple-dose pharmacokinetics of intravenous Peptide 1 (SEQ ID NO. 1) in rats.

| | 5 mg/kg | | 10 mg/kg | | 20 mg/kg |
|---|---|---|---|---|---|
| Parameters | Day 1 | Day 7 | Day 1 | Day 7 | Day 1 |
| Cₘₐₓ (ng/mL) | 7,468 (1,202)*^{,#} | 10,137 (688) | 27,533 (1,419)* | 21,850 (759) | 35,400 (5,350) ^{#} |
| λz (h⁻¹) | 0.056 | 0.104 | 0.148 | 0.133 | 0.221 |
| AUCτ (ng*h/mL) | 14,956 | 12,468 | 41,669 | 34,121 | 126,860 |
| AUC_{0-∞} (ng*h/mL) | 15,948 | 12,704 | 42,085 | 34,688 | 127,386 |
| CL (mL/h/kg) | 334 | 401 | 240 | 293 | 157 |
| MRT (h) | 4.37 | 2.92 | 2.88 | 3.45 | 4.81 |
| Vss (mL/kg) | 1,461 | 1,173 | 691 | 1,011 | 758 |

(Estimate, SE)
Cₘₐₓ, maximum observed plasma concentration;
λz, terminal elimination rate constant;
AUCτ, area under the curve to dosing interval (24 h), AUC_{0-∞}, area under the curve extrapolated to infinity;
CL, clearance;
MRT, mean residence time;
Vss, volume of distribution at steady state
*^{,#}denote statistically significant differences between matching groups (p < 0.05)

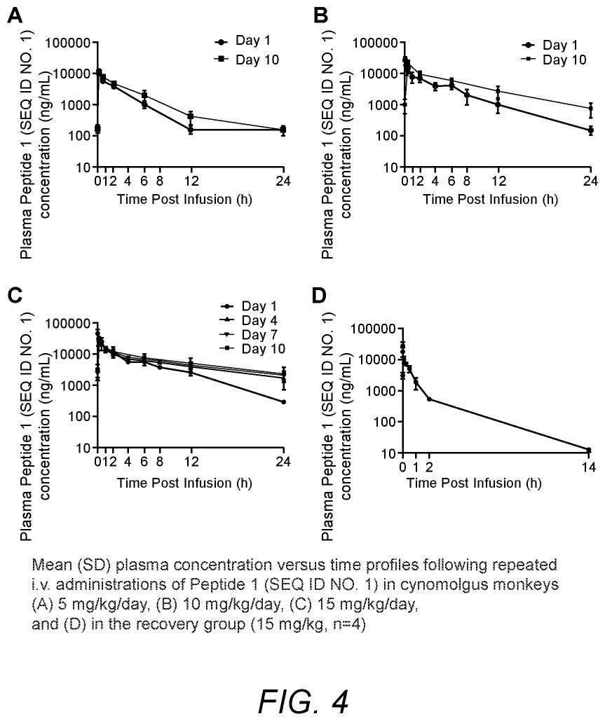

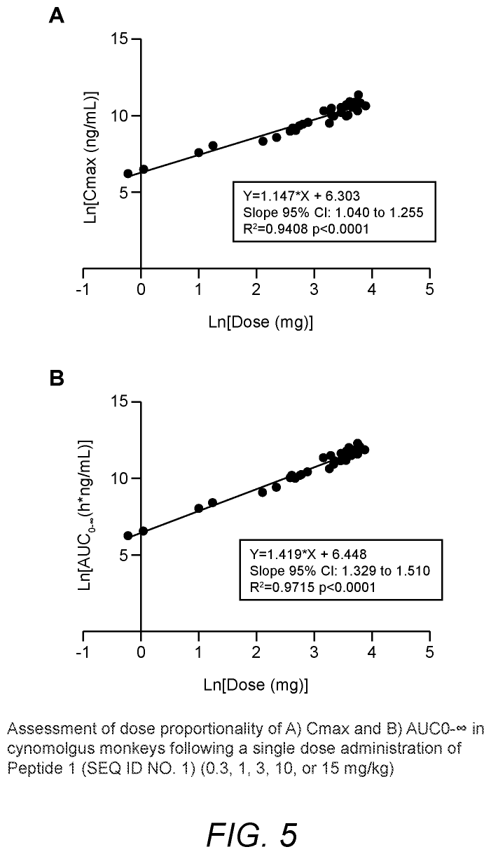

Cynomolgus monkey studies: The mean plasma concentration-time profiles in cynomolgus monkeys following single or multiple dose administrations of Peptide 1 (SEQ ID NO. 1) are illustrated in FIG. 2C and FIGS. 4A-4D, respectively. The corresponding PK parameters calculated by NCA are outlined in Table 3. Of the total 233 infusions from 24 cynomolgus monkeys, four infusions deviated more than 10% from the intended 1-h infusion. However, these deviations did not occur on blood PK sampling days and therefore did not influence the PK analysis. Data from two separate studies involving single and multiple dosing in cynomolgus monkeys were combined in this analysis. Overall, concentration-time profiles displayed a biphasic pattern, with a prolonged elimination phase at higher doses. Plasma concentrations of Peptide 1 (SEQ ID NO. 1) after a single dose of 0.3 or 1 mg/kg were detectable up to 12 h post end of infusion. In the GLP-compliant 10-day TK study, all animals received ten i.v. doses except for one female monkey in 15 mg/kg, which received a total of 9 doses due to issues with venous access. Extended sampling with cynomolgus monkeys assigned to the recovery group (15 mg/kg) revealed that plasma levels of Peptide 1 (SEQ ID NO. 1) were quantifiable on Day 12 (537 ng/mL) and Day 24 (12.5 ng/mL), indicating that Peptide 1 (SEQ ID NO. 1) exhibits a long terminal half-life (FIG. 4D). The average terminal half-life in the recovery animals was 47.2 h when compared with 9.53 h in the main group with the shorter sampling period. The Cmax and AUC_{0-∞} were slightly higher in males compared to females, but the differences did not reach statistical significance. Detailed assessment of dose proportionality in cynomolgus monkeys administered a single i.v. dose ranging from 0.3 to 15 mg/kg revealed that both Cmax and AUC0-∞ increased greater than dose-proportional (see FIGS. 5A and 5B). Specifically, AUC_{0-∞} was dose-proportional at lower doses (0.3-3 mg/kg) but began to deviate from dose proportionality at higher doses (≥5 mg/kg) (data not shown). Dose proportionality assessment at steady-state demonstrated that while the Cmax increased dose proportionally (Cmax, Y=0.9553*X+6.805 [Slope 95% CI: 0.5573 to 1.353, r²=0.5705]), the AUCτ increased greater than dose proportionally (AUCτ, Y=1.422*X+6.745 [Slope 95% CI: 1.072 to 1.772, r²=0.7916]). Comparisons of AUCs after a single dose (Day 1) and repeat dose administrations (Day 10) revealed statistically significant accumulations at 5- and 10 mg/kg, yielding approximately 1.4- and 1.5-fold higher mean AUCτ compared to the mean AUC_{0-∞} on Day 1 for 5- and 10 mg/kg, respectively [5 mg/kg (p=0.0229) and 10 mg/kg (p=0.0103)]. Although there was a trend towards Peptide 1 (SEQ ID NO. 1) accumulation at 15 mg/kg at a steady-state (Day 10), the difference did not reach statistical significance (p=0.1879).

TABLE 3

| | 0.3 mg/kg | 1 mg/kg | 3 mg/kg | 5 mg/kg | |
|---|---|---|---|---|---|
| | Day 1 | Day 1 | Day 1 | Day 1 | Day 10 |
| Parameter | n = 2 | n = 2 | n = 2 | n = 6 | n = 6 |
| Cₘₐₓ | 582 | 2,560 | 4,750 | 10,700 | 11,675 |
| (ng/mL) | (109) | (820) | (905) | (2,455) | (2,036) |
| λz (h⁻¹) | 0.523 | 0.251 | 0.078 | 0.323 | 0.157 |
| | (0.119) | (0.035) | (0.017) | (0.016)^{#} | (0.009)^{#} |
| AUCτ | 591 | 3,818 | 10,334 | 27,160 | 38,936 |
| (ng*h/mL) | (145) | (961) | (2,239) | (4,286) | (9,829) |
| AUC_{0-∞} | 617 | 3,875 | 10,740 | 27,176 | 39,997 |
| (ng*h/mL) | (137) | (973) | (2,485) | (4,292) | (10,219) |
| CL | 498 | 266 | 287 | 188 | 136 |
| (mL/h/kg) | (111) | (66.9) | (66.4) | (29.2)^{#} | (36.5)^{#} |
| MRT_{inf} (h) | 1.24 | 1.82 | 4.30 | 2.94 | 4.84 |
| | (0.16) | (0.07) | (0.60) | (0.15) | (0.57) |
| Vss (mL/kg) | 626 | 486 | 1,215 | 550 | 645 |
| | (218) | (139) | (112) | (74.6) | (123) |

| | 10 mg/kg | | 15 mg/kg | | | |
|---|---|---|---|---|---|---|
| | Day 1 | Day 10 | Day 1 | Day 4 | Day 7 | Day 10 |
| Parameter | n = 8 | n = 6 | n = 12 | n = 2 | n = 2 | n = 9 |
| Cₘₐₓ | 26,675 | 27,333 | 46,458 | 31,100 | 32,850 | 32,233 |
| (ng/mL) | (8,240) | (7,361) | (15,247)^{†} | (283) | (919) | (13,933)^{†} |
| λz (h⁻¹) | 0.192 | 0.120 | 0.149 | 0.072 | 0.071 | 0.078 |
| | (0.034)* | (0.018)* | (0.019)^{†} | (0.020) | (0.017) | (0.020)^{†} |
| AUCτ | 76,083 | 117,529 | 138,135 | 139,595 | 154,149 | 173,315 |
| (ng*h/mL) | (23,562) | (26,077) | (34,942) | (2,366) | (12,406) | (67,934) |
| AUC_{0-∞} | 76,820 | 124,790 | 142,095 | 167,061 | 187,609 | 212,728 |
| (ng*h/mL) | (23,871) | (30,604) | (35,842) | (16,034) | (31,623) | (104,487) |
| CL | 143 | 88.5 | 114 | 107 | 97.6 | 103 |
| (mL/h/kg) | (48.2) | (18.5)* | (25.7) | (1.82) | (7.86) | (53.0) |
| MRT_{inf} (h) | 4.40 | 7.20 | 5.48 | 11.8 | 12.3 | 12.0 |
| | (0.50) | (1.04) | (0.76) | (2.46) | (3.08) | (3.33) |
| Vss | 629 | 624 | 629 | 1,263 | 1,193 | 1,139 |
| (mL/kg) | (231) | (88.2) | (176)^{†} | (242) | (204) | (411)^{†} |

Mean (SD)
Cₘₐₓ, maximum observed plasma concentration;
λz, terminal elimination rate constant;
AUCτ, area under the curve to dosing interval (24 h), AUC_{0-∞}, area under the curve extrapolated to infinity;
CL, clearance;
MRT, mean residence time;
Vss, volume of distribution at steady state
^{#,}*^{,†}denote statistically significant differences within the dosing group between Day 1 and Day 10 (p < 0.05)

Vervet monkey studies: The mean plasma concentration-time profile of Peptide 1 (SEQ ID NO. 1) in the vervet monkeys after a single i.v. bolus administration of Peptide 1 (SEQ ID NO. 1) is shown in FIG. 2D, and the PK parameters are presented in Table 4. Following the bolus administration, plasma concentrations of Peptide 1 (SEQ ID NO. 1) declined monoexponentially. Plasma concentrations below the lower limit of quantification were from the analysis.

TABLE 4

| Parameters | Estimate |
|---|---|
| Cₘₐₓ(ng/mL) | 5,193 |
| λz (h⁻¹) | 0.204 |
| AUCτ (ng*h/mL) | 21,949 |
| AUC_{0-∞} (ng*h/mL) | 22,110 |
| CL (mL/h/kg) | 136 |
| MRT (h) | 4.71 |
| Vss (mL/kg) | 639 |

Cₘₐₓ, maximum observed plasma concentration;
λz, terminal elimination rate constant;
AUCτ, area under the curve to dosing interval (24 h), AUC_{0-∞}, area under the curve extrapolated to infinity;
CL, clearance;
MRT, mean residence time;
Vss, volume of distribution at steady state

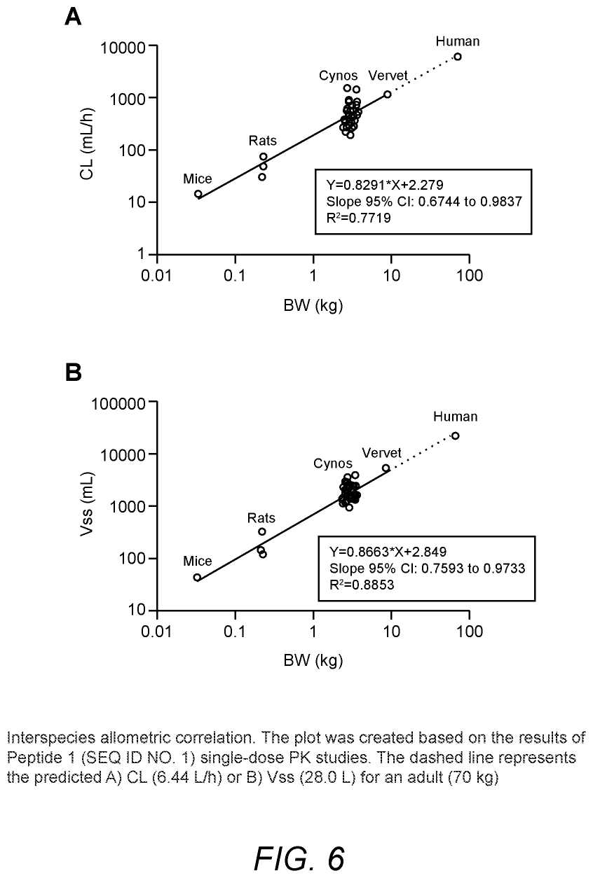

Interspecies allometric scaling: Overall, linear regression of logarithmically transformed CL or Vss against log-transformed body weight (BW) from the four animal species resulted in a reasonable fit, as evidenced by the relatively high r². (FIGS. 6A and 6B). The allometric scaling equations for CL and Vss were Y=190.1·BW^{0.829} (r²=0.7719) and Y=706.3·BW^{0.8663} (r²=0.8853), respectively, which yielded the predicted human CL of 6.44 L/h and volume of distribution at steady state (Vss) of 28.0 L. Based on the target plasma AUC_{0-∞} of approximately 3,869 and 9,001 ng*h/mL, which were previously established in a murine model of endotoxin-induced acute lung injury (ALI), the estimated human equivalent doses (HED) to reach therapeutic efficacy are between 24.9 and 58.0 mg for a 70 kg individual, or 0.36 and 0.83 mg/kg.

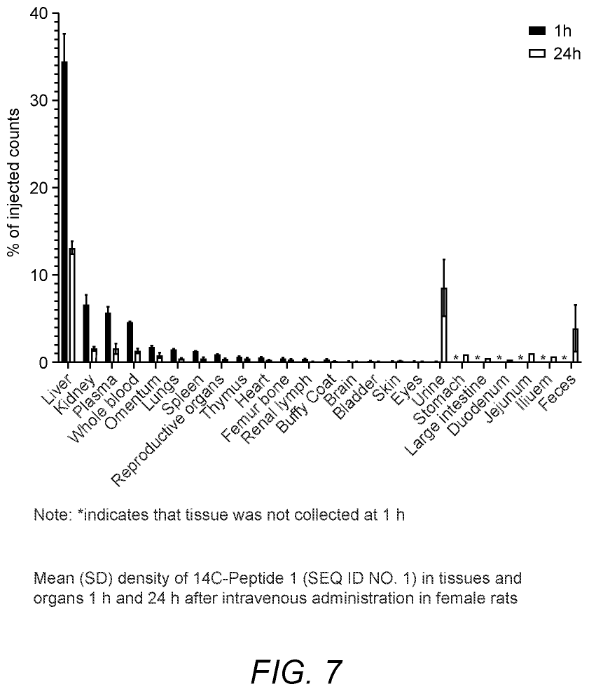

Biodistribution: A biodistribution study was undertaken to determine the patterns of distribution and potential routes of elimination of Peptide 1 (SEQ ID NO. 1) in rats after a single dose i.v. administration of ¹⁴C-Peptide 1 (SEQ ID NO. 1) equivalent to 5 mg/kg. Widespread distribution of ¹⁴C-Peptide 1 (SEQ ID NO. 1) was observed at 1 h, with the highest density measured in the liver, followed by the kidney (see FIG. 7). At 1 h, there was a trace amount of ¹⁴C counts measured in the urine, skin, leg muscle, eyes, and brain. After 24 h, the density of ¹⁴C counts in the tissues and organs decreased compared to counts detected at 1 h, except for in the urine. The ¹⁴C counts in urine increased from trace amounts at 1 h to 8.5% after 24 h. Moreover, approximately 4% of the ¹⁴C-Peptide 1 (SEQ ID NO. 1) dose administered was recovered in the feces at 24 h, suggesting that the major route of elimination is urinary, followed by biliary excretion.

### Safety

Rat studies: In general, single doses up to 10 mg/kg were well-tolerated in male and female rats. During the study, mortality was observed in a total of 7 rats. Specifically, 1 out of 18 female rats in the placebo group was found dead on Day 6, and another 1 out of 16 female rats in the 5 mg/kg dose group was found dead on Day 15. However, the mortality of the female rat in the 5 mg/kg group was determined to be unrelated to Peptide 1 (SEQ ID NO. 1) treatment due to minimal clinical manifestations until the day of death and the timing of the event. Additionally, a single i.v. administration of 20 mg/kg Peptide 1 (SEQ ID NO. 1) led to acute, treatment-related mortality in 5 out of 12 rats on Day 1. Of the five rats, three male rats were found dead, and one male and one female rat were euthanized due to moribund conditions.

Following once daily i.v. infusion of Peptide 1 (SEQ ID NO. 1) (5- and 10 mg/kg/day), non-adverse, treatment-related clinical signs such as muscle fasciculation, lethargy, swollen nose, chin, and/or cheeks, swollen front limbs, reluctance to walk and/or stand, hypoactivity, ataxia, and increased respiration, were noted throughout the study at both dose levels, but were temporary and resolved during the recovery period. There were no significant changes in the body weight in rats except for those in the 20 mg/kg dosing group, where significant decreases in body weights were recorded in both male and female rats (data not shown). Food consumption was not significantly affected by treatment administration in rats at any dose level.

No treatment-related changes in hematological parameters were observed in male and female rats at 5 mg/kg at the end of treatment (Day 8) (Table 5). At the end of recovery, RBC volume distribution width (RDW) was significantly elevated in female rats at 5 mg/kg and was outside of the historical control range (HCR) for female Sprague Dawley rats (data not shown). At 10 mg/kg, a significant decrease in absolute reticulocytes in male rats treated with 10 mg/kg at the end of treatment. However, this value was within the HCR for male Sprague Dawley rats of this age and therefore was considered non-adverse. In female rats, there were significant increases in WBC, and absolute lymphocytes and monocytes at the end of treatment when compared to controls. However, these increases were considered non-adverse due to lack of dose-dependency and the reversibility of the changes by the end of the recovery period (data not shown). At the end of recovery, mean cell hemoglobin (MCH) and mean cell hemoglobin concentration (MCHC) increased modestly in male rats compared to the controls, but the values remained within the reference range for male rats of similar age. At 20 mg/kg, white blood cell counts (WBC), relative and absolute neutrophils, relative and absolute monocytes, and relative and absolute large unclassified cells (LUC) counts were significantly elevated in male rats, while relative lymphocytes, relative and absolute eosinophils, and platelet count significantly decreased on Day 2 compared to the controls at the end of treatment. The relative and absolute neutrophil, relative lymphocyte, and relative and absolute monocyte values were outside of the HCR for male rats. In female rats, there were significant increases in absolute reticulocytes and monocytes, while significant decreases in relative and absolute eosinophils were observed on Day 2 (interim euthanasia) when compared with controls at the end of treatment.

TABLE 5

| | Male | | | | Female | | | |
|---|---|---|---|---|---|---|---|---|
| | 0 | 5 | 10 | 20 | 0 | 5 | 10 | 20 |
| Parameter | mg/kg | mg/kg | mg/kg | mg/kg^{d} | mg/kg | mg/kg | mg/kg | mg/kg^{d} |
| WBC | 7.95 | 8.40 | 10.05 | 13.70 | 6.95 | 6.50 | 10.75 | 8.15 |
| (10³/mm³) | (1.93) | (2.53) | (3.28) | (4.90) ^{a, b} | (2.43) | (3.50) | (3.68) ^{a} | (5.70) |
| Hemoglobin | 13.8 | 14.1 | 14.2 | 12.1 | 13.2 | 13.5 | 13.7 | 15.2 |
| (g/dL) | (0.9) | (1.0) | (0.7) | (1.0) ^{b, c} | (0.9) | (1.0) | (0.4) | (4.6) |
| Hct (%) | 42.6 | 43.8 | 43.7 | 37.3 | 40.8 | 40.5 | 41.2 | 46.9 |
| | (2.9) | (1.7) | (1.2) | (3.7) ^{b, c} | (2.2) | (1.9) | (1.8) | (14.3) |
| RBC (10⁶/mm³) | 6.69 | 7.06 | 7.06 | 6.04 | 6.92 | 6.88 | 7.21 | 7.63 |
| | (0.64) | (0.23) | (0.38) | (0.47) ^{b, c} | (0.36) | (0.33) | (0.40) | (2.19) |
| MCH (pg) | 20.4 | 20.0 | 20.2 | 20.0 | 19.3 | 19.1 | 19.0 | 19.6 |
| | (0.7) | (1.4) | (0.6) | (0.6) | (0.8) | (1.0) | (0.5) | (0.9) |
| MCV (fL) | 62.4 | 61.8 | 62.6 | 62.5 | 58.0 | 57.9 | 57.3 | 61.0 |
| | (3.1) | (2.9) | (3.1) | (2.1) | (2.7) | (2.8) | (2.4) | (2.2) ^{c} |
| MCHC (g/dL) | 32.0 | 32.1 | 32.4 | 31.8 | 33.1 | 33.4 | 33.3 | 32.3 |
| | (0.8) | (0.3) | (0.7) | (0.5) | (0.9) | (0.4) | (0.4) | (0.3) ^{b, c} |
| RDW (%) | 14.1 | 14.0 | 13.1 | 14.3 | 12.5 | 12.7 | 13.0 | 12.2 |
| | (1.5) | (1.2) | (1.1) | (1.2) | (0.3) | (0.8) | (0.4) | (0.6) ^{c} |
| HDW (g/dL) | 2.27 | 2.30 | 2.20 | 2.44 | 2.37 | 2.37 | 2.45 | 2.37 |
| | (0.29) | (0.09) | (0.20) | (0.25) ^{c} | (0.19) | (0.16) | (0.16) | (0.32) |
| Neutrophils (%) | 17.4 | 20.2 | 22.6 | 61.1 | 22.9 | 26.7 | 19.9 | 33.6 |
| | (6.0) | (4.7) | (7.9) | (6.1) ^{a, b, c} | (16.0) | (11.1) | (5.5) | (14.5) |
| Lymphocytes | 75.6 | 71.8 | 70.9 | 30.9 | 69.8 | 68.1 | 72.0 | 60.5 |
| (%) | (7.8) | (5.4) | (11.2) | (4.7) ^{a, b, c} | (18.6) | (8.7) | (4.4) | (12.9) |
| Monocytes (%) | 2.5 | 2.9 | 2.7 | 8.1 | 3.1 | 2.6 | 3.5 | 3.8 |
| | (1.3) | (1.6) | (1.1) | (2.6) ^{a, b, c} | (1.3) | (1.0) | (1.4) | (1.5) |
| Eosinophils (%) | 3.3 | 3.9 | 3.0 | 1.1 | 4.1 | 3.6 | 3.3 | 1.5 |
| | (0.5) | (1.3) | (1.9) | (0.3) ^{a, b, c} | (2.5) | (2.4) | (1.0) | (0.5) ^{a, b, c} |
| Basophils (%) | 0.3 | 0.3 | 0.3 | 0.2 | 0.3 | 0.3 | 0.4 | 0.5 |
| | (0.1) | (0.1) | (0.2) | (0.1) | (0.2) | (0.2) | (0.2) | (0.5) |
| LUC (%) | 0.3 | 0.3 | 0.3 | 0.5 | 0.3 | 0.3 | 0.35 | 0.5 |
| | (0.2) | (0.1) | (0.2) | (0.5) ^{a} | (0.1) | (0.1) | (0.28) | (0.3) |
| Reticulocytes | 5.8 | 5.2 | 4.6 | 6.4 | 4.0 | 3.9 | 4.1 | 5.3 |
| (%) | (1.4) | (0.4) | (0.8) | (1.6) ^{b, c} | (1.2) | (1.0) | (0.6) | (1.4) |
| Neutrophil | 1.48 | 2.04 | 1.85 | 8.40 | 1.75 | 1.33 | 2.18 | 2.55 |
| absolute | (0.83) | (0.96) | (0.61) | (3.42) ^{a, b, c} | (0.77) | (0.99) | (1.30) | (2.72) |
| (10³/mm³) | | | | | | | | |
| Lymphocyte | 5.49 | 6.03 | 6.69 | 4.24 | 4.40 | 4.78 | 7.13 | 5.56 |
| absolute | (2.00) | (1.81) | (2.92) | (1.79) ^{b, c} | (1.75) | (3.21) | (1.76) ^{a} | (2.93) |
| (10³/mm³) | | | | | | | | |
| Monocyte | 0.19 | 0.22 | 0.28 | 0.93 | 0.17 | 0.17 | 0.39 | 0.33 |
| absolute | (0.16) | (0.20) | (0.10) | (0.29) ^{a, b, c} | (0.09) | (0.08) | (0.13) ^{a, b} | (0.26) ^{a} |
| (10³/mm³) | | | | | | | | |
| Eosinophils | 0.27 | 0.37 | 0.31 | 0.15 | 0.28 | 0.30 | 0.33 | 0.13 |
| absolute | (0.05) | (0.10) | (0.24) | (0.06) ^{a, b, c} | (0.22) | (0.23) | (0.14) | (0.06) ^{a, c} |
| (10³/mm³) | | | | | | | | |
| Basophils | 0.02 | 0.03 | 0.03 | 0.03 | 0.02 | 0.02 | 0.04 | 0.06 |
| absolute | (0.01) | (0.02) | (0.02) | (0.03) | (0.03) | (0.02) | (0.02) | (0.06) |
| (10³/mm³) | | | | | | | | |
| LUC absolute | 0.02 | 0.03 | 0.03 | 0.07 | 0.02 | 0.02 | 0.04 | 0.05 |
| (10³/mm³) | (0.01) | (0.02) | (0.02) | (0.05) ^{a, b, c} | (0.03) | (0.03) | (0.03) | (0.03) |
| Reticulocytes | 378.3 | 359.7 | 314.2 | 373.3 | 277.8 | 256.7 | 277.3 | 326.0 |
| absolute | (90.6) | (38.0) | (33.4) ^{a} | (106.5) ^{c} | (60.6) | (53.5) | (42.0) | (60.4) ^{a, b} |
| (10³/mm³) | | | | | | | | |
| Platelets | 895 | 1,052 | 1,086 | 728 | 822 | 801 | 939 | 604 |
| (10³/mm³) | (93) | (145) | (192) | (86) ^{a, b, c} | (169) | (306) | (315) | (524) ^{c} |

Data represents median (IQR)
WBC, White blood cell count; RBC, Red blood cell count; RDW, RBC volume distribution width; HDW, Hemoglobin concentration distribution width; MCH, Mean cell hemoglobin; MCHC, Mean cell hemoglobin concentration; MCV, Mean cell volume; LUC, Large unclassified cells.
^{a} p < 0.05 compared with control (0 mg/kg)
^{b} p < 0.05 compared with 5 mg/kg
^{c} p < 0.05 compared with 10 mg/kg
^{d}Measurement on day of unscheduled euthanasia (Day 2), excludes animals found dead

There were no significant treatment-induced abnormalities in serum biochemical parameters in male and female rats that received 5 or 10 mg/kg/day at the end of treatment (Table 6). At the end of recovery, glucose levels were slightly elevated in female rats at 10 mg/kg (data not shown). At the highest dose examined (20 mg/kg), chloride and albumin levels significantly decreased while serum urea nitrogen (Urea N) levels increased in male rats on Day 2 compared to controls. In female rats, alanine aminotransferase (ALT), calcium, and gamma-glutamyl transferase (GGT) levels were significantly elevated compared to controls. In both male and female rats, albumin/globulin ratio (A/G), creatinine, glucose, triglyceride levels were significantly elevated while total protein (TP), sodium (Na), and globulin levels significantly reduced when compared with controls. Albumin, A/G, globulin, Urea N, TP, and triglyceride levels were outside the HCR in male rats and A/G, GGT, globulin, glucose, TP, and triglycerides were outside the HCR in female rats. The remaining parameters were within the HCR and therefore considered non-adverse.

TABLE 6

| | Male | | | | Female | | | |
|---|---|---|---|---|---|---|---|---|
| | 0 | 5 | 10 | 20 | 0 | 5 | 10 | 20 |
| Parameter | mg/kg | mg/kg | mg/kg | mg/kg^{d} | mg/kg | mg/kg | mg/kg | mg/kg^{d} |
| ALT (U/L) | 42.5 | 40.0 | 32.5 | 74.0 | 33.0 | 35.0 | 32.5 | 62.0 |
| | (5.5) | (9.8) | (4.5) | (38.0) ^{b, c} | (8.0) | (7.0) | (6.0) | (28.5) ^{a, b, c} |
| Albumin | 3.7 | 3.8 | 3.7 | 2.7 | 3.6 | 3.9 | 3.9 | 3.0 |
| (g/dL) | (0.2) | (0.0) | (0.2) | (0.0) ^{a, b, c} | (0.2) | (0.3) | (0.1) | (0.6) ^{b, c} |
| A/G | 2.4 | 2.2 | 2.35 | 2.70 | 2.20 | 2.10 | 2.15 | 3.00 |
| | (0.4) | (0.3) | (0.18) | (0.10) ^{a, b, c} | (0.10) | (0.20) | (0.18) | (0.40) ^{a, b, c} |
| ALP (U/L) | 248.5 | 212.0 | 212.5 | 232.0 | 124.0 | 92.0 | 109.5 | 142.0 |
| | (72.8) | (40.3) | (51.5) | (45.0) | (28.0) | (21.0) | (30.5) | (34.5) ^{b, c} |
| AST (U/L) | 158.5 | 133.0 | 113.5 | 245.0 | 149.0 | 139.0 | 122.0 | 397.0 |
| | (45.8) | (34.0) | (16.8) | (100.0) ^{b, c} | (22.0) | (35.0) | (14.3) | (345.5) ^{b, c} |
| Calcium | 9.60 | 9.75 | 9.70 | 9.40 | 9.60 | 9.80 | 9.95 | 10.30 |
| (mg/dL) | (0.18) | (0.45) | (0.20) | (0.50) ^{b} | (0.20) | (0.20) | (0.28) | (0.60) ^{a, b} |
| Chloride | 105.0 | 104.5 | 105.0 | 101.0 | 105.0 | 105.0 | 105.0 | 104.0 |
| (mmol/L) | (1.8) | (1.8) | (1.0) | (1.0) ^{a, b, c} | (3.0) | (2.0) | (2.0) | (3.0) |
| Cholesterol | 59.5 | 70.5 | 66.0 | 67.0 | 52.0 | 55.0 | 55.0 | 52.0 |
| (mg/dL) | (13.0) | (10.3) | (14.3) | (7.0) | (7.0) | (15.0) | (26.5) | (6.5) |
| CK (U/L) | 828 | 766 | 613 | 707 | 914 | 792 | 474 | 1,506 |
| | (342) | (396) | (255) | (615) | (450) | (198) | (211) | (2,605) |
| Creatinine | 0.2 | 0.2 | 0.3 | 0.4 | 0.3 | 0.3 | 0.3 | 0.5 |
| (mg/dL) | (0.0) | (0.1) | (0.1) | (0.1) ^{a, b, c} | (0.0) | (0.0) | (0.0) | (0.2) ^{a, b, c} |
| GGT (U/L) | 0.0 | 0.0 | 0.0 | 0.0 | 0.0 | 0.0 | 0.0 | 4.0 |
| | (0.0) | (0.0) | (0.0) | (0.0) | (0.0) | (0.0) | (0.0) | (5.5) ^{a, b, c} |
| Globulin | 1.6 | 1.7 | 1.6 | 1.0 | 1.7 | 1.8 | 1.8 | 1.0 |
| (g/dL) | (0.2) | (0.2) | (0.2) | (0.0) ^{a, b, c} | (0.1) | (0.1) | (0.2) | (0.3) ^{a, b, c} |
| Glucose | 87 | 73 | 83 | 175 | 105 | 97 | 99 | 218 |
| (mg/dL) | (7) | (16) | (7) | (35) ^{a, b, c} | (7) | (16) | (6) | (70) ^{a, b, c} |
| Phos (mg/dL) | 8.25 | 8.45 | 8.6 | 9.3 | 7.6 | 7.3 | 7.05 | 9.3 |
| | (0.75) | (0.55) | (1.0) | (3.8) | (0.7) | (0.8) | (0.75) | (5.0) |
| K (mmol/L) | 4.8 | 5.15 | 5.1 | 5.1 | 4.4 | 4.9 | 5.05 | 4.9 |
| | (0.1) | (0.18) | (0.3) | (0.6) | (0.4) | (0.4) | (0.45) | (0.65) |
| Urea N | 17.5 | 16.5 | 19.0 | 28 | 20 | 20 | 21 | 20 |
| (mg/dL) | (4.25) | (4.0) | (3.5) | (6) ^{a, b, c} | (1) | (2) | (1.8) | (3) |
| Na (mmol/L) | 145 | 144 | 144 | 140 | 145 | 143 | 142.5 | 141 |
| | (1.75) | (1.8) | (0) | (2) ^{a, b, c} | (3) | (1) | (1.0) | (2.5) ^{a} |
| T-Bil | 0.00 | 0.00 | 0.00 | 0.10 | 0.00 | 0.00 | 0.00 | 0.00 |
| (mg/dL) | (0.00) | (0.00) | (0.00) | (0.20) | (0.00) | (0.00) | (0.00) | (0.00) |
| TP (g/dL) | 5.2 | 5.5 | 5.35 | 3.7 | 5.4 | 5.8 | 5.7 | 3.90 |
| | (0.25) | (0.18) | (0.28) | (0.1) ^{a, b, c} | (0.4) | (0.5) | (0.4) | (0.85) ^{a, b, c} |
| Triglycerides | 29 | 37 | 29 | 193 | 22 | 25 | 26 | 106 |
| (mg/dL) | (12) | (22) | (5) | (111) ^{a, b, c} | (4) | (7) | (12) | (37) ^{a, b, c} |

Data represents median (IQR)
ALT, alanine aminotransferase; A/G, Albumin/Globulin ratio; ALP, Alkaline phosphatase; AST, Aspartate aminotransferase; CK, Creatine kinase; GGT, Gamma glutamyltransferase; Phos, inorganic phosphorus; K, potassium; Na, Sodium; Urea N, serum Urea nitrogen; T-Bil, total bilirubin; TP, total protein.
^{a} p < 0.05 compared with control (0 mg/kg)
^{b} p < 0.05 compared with 5 mg/kg
^{c} p < 0.05 compared with 10 mg/kg
^{d}Measurement on day of unscheduled euthanasia (Day 2), excludes animals found dead

There were no treatment-related changes in coagulation parameters in male and female rats at 5 or 10 mg/kg group at the end of treatment (Table 7) or the end of recovery (data not shown). However, at 20 mg/kg, the prothrombin time (PT) and activated partial thromboplastin time (APTT) were significantly elevated in both male and female rats, while fibrinogen levels decreased in male rats when compared to controls at the end of treatment. Of these, fibrinogen levels were outside of HCR in male rats, and both PT and APTT were outside of HCR in female rats.

TABLE 7

| | Male | | | | Female | | | |
|---|---|---|---|---|---|---|---|---|
| | 0 | 5 | 10 | 20 | 0 | 5 | 10 | 20 |
| Parameter | mg/kg | mg/kg | mg/kg | mg/kg^{d} | mg/kg | mg/kg | mg/kg | mg/kg^{d} |
| PT | 16.3 | 16.0 | 17.5 | 21.3 | 16.3 | 16.3 | 17.1 | 23.5 |
| (seconds) | (1.6) | (0.5) | (1.4) | (1.4) ^{a, b} | (0.7) | (1.4) | (0.6) | (9.8) ^{a, b} |
| APTT | 14.1 | 14.8 | 14.6 | 18.5 | 10.5 | 10.6 | 10.9 | 29.2 |
| (seconds) | (2.9) | (1.0) | (1.1) | (3.1) ^{a, b, c} | (1.9) | (0.7) | (2.4) | (20.7) ^{a, b, c} |
| Fibrinogen | 264 | 263 | 261 | 98 | 269 | 223 | 283 | 83 |
| (mg/dL) | (50) | (9) | (55) | (19) ^{a, b, c} | (68) | (80) | (37) | (152) ^{c} |

Data represents median (IQR)
PT, Prothrombin time; APTT, Activated partial thromboplastin time.
^{a} p < 0.05 compared with control (0 mg/kg)
^{b} p < 0.05 compared with 5 mg/kg
^{c} p < 0.05 compared with 10 mg
^{d}Measurement on day of unscheduled euthanasia (Day 2), excludes animals found dead.

There were no treatment-related changes in urinalysis parameters from male and female rats receiving 5 or 10 mg/kg (data not shown). Due to early removal, overnight urine samples were not collected in rats in the 20 mg/kg group and therefore could not be assessed. No treatment-related abnormalities were detected during the post-exposure ophthalmology assessments with any dosing group. Histopathological evaluations of rats in the 20 mg/kg group showed discolorations in the kidney, brain, and lungs, with bronchi and trachea filled with fluids at interim euthanasia. There were no macroscopic findings related to Peptide 1 (SEQ ID NO. 1) treatment in any dose groups at interim euthanasia (for 20 mg/kg group, Day 2), terminal euthanasia (Day 8), or recovery euthanasia (Day 24). Key treatment-related microscopic findings included non-adverse, dose-dependent increased incidence of minimal to mild liver necrosis in female rats administered 5- and 10 mg/kg/day at the end of treatment. However, liver necrosis was not present in female animals (5- and 10 mg/kg/day) euthanized at the end of the recovery period and did not lead to increases in parameters included in the liver function panels (TP, albumin, total bilirubin, and liver enzymes). Therefore, these observations were considered recoverable and non-adverse. Adverse treatment-related severity ranging from minimal to moderate liver necrosis, defined by focal to multifocal areas of lytic to coagulative necrosis, was observed in male rats administered 20 mg/kg. Mild to severe adrenal necrosis, characterized by unilateral to bilaterally coagulative cortical to corticomedullar necrosis, was observed in both male and female rats administered 20 mg/kg.

Based on the lack of adverse clinical signs or abnormalities in parameters at the 10 mg/kg dose, the no-observed-adverse-effect-level (NOAEL) was established at 10 mg/kg/day in rats. The lowest observed adverse effect level (LOAEL), which the FDA defines as the lowest dose tested in preclinical species with adverse effects, was established at 20 mg/kg in rats.

Cynomolgus monkey studies: Overall, Peptide 1 (SEQ ID NO. 1) at doses up to 15 mg/kg was well tolerated in male and female monkeys. In the non-GLP dose range-finding study, all animals survived the study without any treatment-related adverse clinical signs. The maximum tolerated dose (MTD) was established at 15 mg/kg based on these data. Similarly, in the GLP-compliant 10-day repeat dose TK study, no mortality or unscheduled euthanasia occurred at any dose level. In addition, no significant changes in body weight were observed in the monkeys. Treatment-related decrease in food consumption was observed in females at 10 mg/kg/day and in both sexes at 15 mg/kg/day during the dosing phase, but this was considered non-adverse as the animals recovered to baseline by the end of recovery and the changes in food consumption did not translate to changes in body weight or adverse clinical observations.

Non-adverse treatment-related hematological changes at the end of treatment included statistically significant, but modest increases in absolute LUC counts in male monkeys and absolute monocytes in female monkeys at 15 mg/kg when compared to the respective controls (data not shown). However, the increase in absolute LUC was resolved by the end of recovery and therefore considered non-adverse (data not shown), and the elevated absolute monocyte counts observed in female monkeys were within the HCR for female cynomolgus monkeys. Most notable non-adverse, but significant treatment-related changes in serum biochemical parameters included a slight reduction in inorganic phosphorus (Phos) level and an elevated glucose level in female monkeys at 15 mg/kg/day at the end of treatment. However, these changes were considered non-adverse regardless of statistical significance due to the small magnitude in change, and the elevated values were still within the HCR for female cynomolgus monkeys. A trend towards an increase in fibrinogen levels was noted in all treated monkeys when compared to the control group and baseline levels, but the changes did not reach statistical significance and returned to baseline by the end of the recovery period (data not shown). There were no treatment-related changes in urinalysis parameters or abnormalities in post-exposure ophthalmologic assessments or the electrocardiogram from cynomolgus monkeys at any dose level (data not shown). The lack of significant alterations in clinical pathology parameters in any of the animals with doses up to 15 mg/kg corroborates the results from the non-GLP dose range study in cynomolgus monkeys, which also established an MTD of 15 mg/kg based on no adverse effects on mortality, clinical observations, or bodyweight with up to 15 mg/kg intravenous Peptide 1 (SEQ ID NO. 1) treatment.

Procedure-related gross observations were recorded at the injection sites of three animals at 15 mg/kg, which included abrasions and abnormal texture likely due to repeat catheterization. However, there were no macroscopic findings related to treatment at the end of treatment or recovery. In the main group, microscopic evaluations revealed treatment-related thrombosis at the injection sites at the end of treatment, although there was a lack of a dose trend in incidence and/or severity. In the recovery group, treatment-related thrombosis at the injection site, acute inflammation, edema, hemorrhage, and fibrosis was present in animals receiving 15 mg/kg at the end of recovery. However, comparable observations were also present at the injection sites of control animals in the recovery group, which include minimal to mild injection site fibrosis and mild chronic thrombosis, suggesting that these findings are procedure-related. Lastly, minimal to mild intravascular thrombosis (thromboembolism) was observed in the lung of only the treated animals (males administered ≥10 mg/kg/day and females administered ≥5 mg/kg/day) at the end of treatment (Day 11). The thrombi within the lungs contained varying numbers of inflammatory cells both within the thrombi and in the surrounding connective tissues, and thrombi in the lungs were present predominately in mid to small arteries and capillaries in the alveolar walls with two animals. Thromboembolism was concluded to be related to the peptide administration due to the composition of the thrombi found in the lung, which were likely embolic from the thrombi formed at the injection site. These findings were also resolved by the end of the recovery period, as thrombosis was not identified in any lung sections in the control group or monkeys administered 15 mg/kg.

The summary of the NOAEL and LOAEL determined in each species is listed in Table 8. Given that the repeat administration of Peptide 1 (SEQ ID NO. 1) of up to 15 mg/kg/day was well tolerated in both sexes, NOAEL was established at 15 mg/kg/day in cynomolgus monkeys. LOAEL could not be determined in cynomolgus monkeys as the highest dose examined in the GLP study was well tolerated. Based on this analysis, the HEDs that are equivalent to the NOAEL and LOAEL in rats are 3.1 mg/kg and 11.7 mg/kg for a 70 kg individual, and the HED that is equivalent to NOAEL in cynomolgus monkeys is 15.9 mg/kg for a 70 kg individual.

TABLE 8

| | | NOAEL | LOAEL | |
|---|---|---|---|---|
| | Study | (mg/kg/ | (mg/kg | Adverse effects observed |
| Species | duration | day) | /day) | at the LOAEL |
| Sprague- | 7 days | 10 | 20 | Cold to touch |
| Dawley rats | | | | Laying on side |
| | | | | Abnormal body color (pale) |
| | | | | Unable to walk |
| | | | | Extreme dehydration |
| | | | | Tremors |
| | | | | Labored respiration |
| | | | | Brain, kidney, and lung |
| | | | | discolorations |
| | | | | Enlarged salivary glands |
| Cynomolgus | 7 days | 15 | ND | Not attained |
| monkeys | | | | |
| Cynomolgus | 10 days | 15 | ND | Not attained |
| monkeys | | | | |

ND—Not determined
Safety

Male and female Sprague Dawley rats (n=16-21/sex) were evaluated for potential toxicity for seven day repeat administration of Peptide 1 (SEQ ID NO. 1) and reversibility of any findings. Rats were divided into 3 subgroups within each dosing group: main (n=10/sex), recovery (n=0-5/sex), and toxicokinetics (TK) (n=3-6/sex).

Mortality and clinical observations: Detailed clinical observations were recorded weekly, from a week prior to the study initiation and throughout the study including the day of necropsy in all rats. In cynomolgus monkeys, detailed clinical observations were recorded once at pre-dose and weekly following the study initiation, including the day of necropsy. All animals were observed/monitored for mortality twice daily beginning upon the arrival through release.

Body weight and food consumption: Individual body weight was recorded once at pre-dose and weekly following the initiation of dosing in all rats and three times at pre-dose and at least once weekly following the initiation of dosing in cynomolgus monkeys. Food consumption was quantitatively measured, per cage, once weekly starting on Day 1 and throughout the study in rats and assessed once daily, throughout the study in cynomolgus monkeys.

Hematology, blood chemistry, and coagulation: Blood samples for hematology, coagulation and clinical chemistry were collected from retro-orbital sinus on days of scheduled (day 8, 24, and 25 in rats belonging to the main, recovery and TK group, respectively) or unscheduled necropsy in rats. Blood samples were collected by venipuncture at pre-dose and at the end of treatment (Day 11) in cynomolgus monkeys, and additionally at the end of recovery in a few subset of male and female monkeys in the placebo (n=2/sex) and 15 mg/kg group (n=2-3/sex).

Urinalysis: Overnight urine samples were collected prior to euthanasia in rats, and at pre-dose, Day 10 and end of recovery in cynomolgus monkeys.

Ophthalmology: Ophthalmological examinations, which consisted of funduscopic (indirect ophthalmoscopy) and biomicroscopic (slit lamp) examinations, were performed at pre-dose and Day 6 in rats and at pre-dose and on Day 8 in cynomolgus monkeys.

Electrocardiogram (ECG): ECG was collected at pre-dose, Day 8 within 10 min following the end of infusion and 2 days prior to necropsy in cynomolgus monkeys.

Methods

The pharmacokinetics and safety of intravenous (i.v.) Peptide 1 (SEQ ID NO. 1) were studied in mice, rats, cynomolgus monkeys, and a vervet monkey. Lyophilized Peptide 1 (SEQ ID NO. 1) (purity >98%) was dissolved in filter-sterilized saline solution and used for injections of mice and one vervet monkey. Peptide 1 (SEQ ID NO. 1) solutions employing rats and cynomolgus monkeys were prepared by dilution of formulated Peptide 1 (SEQ ID NO. 1) (12.5 mg/mL in 1% propylene glycol, 20 mM sodium acetate, pH 6.5) in filter-sterilized saline. The summary of study design and schedule of safety assessments are listed in Tables 9 and 10, respectively. The studies included single- and multiple-dose-ranging experiments. All protocols received local IACUC approval before the initiation of the studies.

TABLE 9

| | | | | | Administration | Number of |
|---|---|---|---|---|---|---|
| Species | Study type | Regimen | Dose | | route | animals |
| Mouse | PK study | Single- | 5 | mg/kg | IV bolus | 24 |
| | | dose | | | | |
| Rat | ¹⁴C-Peptide 1 (SEQ ID | Single- | 5 | mg/kg | IV bolus | 5 |
| | NO. 1) biodistribution | dose | | | | |
| | study | | | | | |
| | GLP toxicity study | Single- | 20 | mg/kg | 20 min-IV | 12 |
| | | dose | | | infusion | |
| | | Multiple- | 5 mg/kg | | 20 min-IV | 12 |
| | | dose | daily × 7 | | infusion | 12 |
| | | | days | | | |
| | | | 10 mg/kg | | | |
| | | | daily × 7 | | | |
| | | | days | | | |
| Monkey | Non-GLP dose- | Single- | 0.3 | mg/kg | 1 h-IV | 2 |
| | escalation study | dose | 1 | mg/kg | infusion | 2 |
| | | | 3 | mg/kg | | 2 |
| | | | 10 | mg/kg | | 2 |
| | | Multiple- | 15 mg/kg | | 1 h-IV | 2 |
| | | dose | daily × 7 | | infusion | |
| | | | days | | | |
| | GLP toxicity study | Multiple- | 5 mg/kg | | 1 h-IV | 6 |
| | | dose | daily × 10 | | infusion | 6 |
| | | | days | | | 10 |
| | | | 10 mg/kg | | | |
| | | | daily × 10 | | | |
| | | | days | | | |
| | | | 15 mg/kg | | | |
| | | | daily × 10 | | | |
| | | | days | | | |
| Vervet | A pilot safety study | Single | 3 | mg/kg | IV bolus | 1 |

dose

TABLE 10

| | | | | A non-GLP PK | A GLP 10-day toxicity | |
|---|---|---|---|---|---|---|
| | A GLP 7-Day toxicity | | | study in | study in cynomolgus | |
| | study in rats | | | cynomolgus | monkeys | |
| Safety parameters | Main | Recovery | TK | monkeys | Main | Recovery^{c} |
| Individual | Pre-dose and weekly | | | Pre-dose, Day 1 | Pre-dose and weekly | |
| bodyweight | | | | and 3 | | |
| Mortality | Twice daily | | | Twice daily | Twice daily | |
| Clinical | Weekly | | | Pre-dose, Day 1 | Pre-dose and weekly | |
| observations | | | | and 3 | | |
| Food consumption | Once weekly | | | ND | Daily | |
| Clinical pathology^{a} | Day 8 | Day 24 | Day 25 | Day 6^{d,e}, 8^{d,e}, | Predose, | Pre-dose, |
| | | | | 11^{d,e}, and 15^{d,f} | Day 10 | Day 10 and |
| | | | | | | 23/24 |
| Urinalysis | Day 8 | Day 24 | Day 25 | ND | Predose, | Pre-dose, |
| | | | | | Day 10 | Day 10 and |
| | | | | | | 23/24 |
| Ophthalmology | Pre-dose, Day 6 | | | ND | Pre-dose, Day 8 | |
| Electrocardiogram | ND | | | ND | Pre-dose, | Pre-dose, |
| | | | | | Day 8 | Day 8 and |
| | | | | | | 21/22 |
| Gross pathology^{b} | Day 8 | Day 24 | ND | ND | Day 11 | Day 23/24 |
| Organ weights^{b} | Day 8 | Day 24 | ND | ND | Day 11 | Day 23/24 |
| Microscopic | Day 8 | Day 24 | ND | ND | Day 11 | Day 23/24 |
| pathology^{b} | | | | | | |

ND—Not determined
^{a}Includes hematology, serum chemistry, and coagulation
^{b}Any animals found dead or pre-terminally euthanized are assessed at the time of necropsy
^{c}Includes a subset of cynomolgus monkeys in the main group that received 0 mg/kg (placebo) or 15 mg/kg of intravenous Peptide 1
^{d}Includes cynomolgus monkeys that received seven (7) daily doses of 15 mg/kg of intravenous Peptide 1 (SEQ ID NO. 1)
^{e}Blood collected for hematology and serum chemistry
^{f}Blood collected for coagulation
Pharmacokinetics

Mouse studies: All procedures and protocols involving the use of animals were reviewed and approved by the University of Southern California (USC) IACUC (protocol #20538). Briefly, male (31.7-37.7 g) and female (25.2-35.6 g) CD-1 mice (Charles River Laboratories) were administered a single 5 mg/kg i.v. bolus injection of Peptide 1 (SEQ ID NO. 1) into the lateral tail vein. A total of 24 mice were separated into six groups (n=2/sex/group), with each group assigned to a single, pre-determined time point. Blood samples were collected at 0.25, 1, 2, 4, 8, and 24 h post-dose via terminal cardiac puncture. The collected samples were centrifuged to separate the plasma and stored at −80° C. until analysis.

Rat studies: Pharmacokinetics of Peptide 1 (SEQ ID NO. 1) in Sprague-Dawley rats was evaluated as part of a Good Lab Practice (GLP) 7-day toxicity study. The study protocol was reviewed and approved by the Citoxlab USA IACUC and was conducted in accordance with guidelines from the USA National Research Council. On the day of the dosing (Day 1), male (229-272 g) and female (186-214 g) rats (n=6/sex/group) were assigned to receive repeated doses of 0 (placebo), 5, 10 or 20 mg/kg of Peptide 1 (SEQ ID NO. 1) once daily for 7 days via i.v. infusion (20 min±3 min). The intravenous route was selected as this is the intended route of administration for prophylactic treatment of microbial infection. The doses were chosen based on a pilot single dose-escalation study in rats, which established the MTD of 20 mg/kg (data not shown). Two subgroups of rats with alternating blood sampling schemes (n=3/sex/group) were assigned as follows: subgroup A with blood collections at 0 (pre-dose), 0.5, 6, and 24 h post-infusion and subgroup B with blood collections at 0.083, 2, and 12 h post-infusion. The 24 h post-infusion samples on Day 1 were taken before the administration of the dose on Day 2. Serial blood samples were collected into K2EDTA tubes at the above-mentioned time points on Days 1 and 7 and once on Day 25 (recovery). The samples were centrifuged at 2,700 g for 10 min at 5° C. to separate plasma from the blood and stored at −80° C. until analysis.

Cynomolgus monkey studies: A non-GLP dose range-finding PK study and a GLP 10-day toxicity study were conducted in cynomolgus monkeys (Macaca fascicularis). The study protocol was reviewed and approved by the Citoxlab USA IACUC and was conducted in accordance with guidelines from the USA National Research Council. The non-GLP dose range-finding study included a single ascending dose and a multiple-dose evaluation. In the single ascending dose PK study, male (3.49-3.57 kg) and female (2.66-2.79 kg) cynomolgus monkeys (n=1/sex/group) were randomly assigned to one of two dose groups. Group 1 received a single dose of 0.3 mg/kg Peptide 1 (SEQ ID NO. 1) as an i.v. infusion (60 min±5 min) on Day 1 and a single dose of 3 mg/kg Peptide 1 (SEQ ID NO. 1) on Day 3, while Group 2 received a single dose of 1 mg/kg Peptide 1 (SEQ ID NO. 1) on Day 1 and a single dose of 10 mg/kg Peptide 1 (SEQ ID NO. 1) on Day 3. The doses used in this study are based on a pilot study in cynomolgus monkeys, which demonstrated the safety of single i.v. doses up to and including 10 mg/kg (data not shown). Blood samples were collected into K₂EDTA tubes at the following time points: pre-dose and approximately 0.083, 0.25, 0.5, 1, 2, 4, 8, 12, and 24 h post end of infusion, on Days 1 and 3. In the multiple-dose study, repeated doses of 15 mg/kg Peptide 1 (SEQ ID NO. 1) were administered once daily for seven days via i.v. infusion (60 min±5 min) (n=1 per sex). Serial blood samples were collected into K₂EDTA at pre-dose, and at approximately 0.083, 0.25, 0.5, 1, 2, 4, 8, 12, and 24 h post end of infusion, on Days 1, 4, and 7. The 24 h post-infusion samples on Days 1 and 4 were taken before the administration of the dose on Days 2 and 5, respectively. In the GLP 10-day toxicity study, PK was evaluated following multiple ascending doses of Peptide 1. Male (2.52-3.65 kg) and female (2.39-3.18 kg) cynomolgus monkeys (n=3-5/sex/group) received placebo (sterile saline) or 5, 10 or 15 mg/kg of Peptide 1 (SEQ ID NO. 1) once daily for 10 days via i.v. infusion (60 min±5 min). Serial blood samples were collected into K₂EDTA tubes at the following time points: pre-dose, 0.083, 0.5, 2, 6, and 12 h post-infusion on Day 1 and pre-dose, 0.083, 0.5, 2, 6, 12, and 24 h post-infusion on Day 10. Additional blood samples were collected on Days 12 and 24 from monkeys that received 0 or 15 mg/kg (n=2/sex/group). All samples were centrifuged at ˜2700×g for 10 min at −5° C. to isolate plasma and stored at −80° C. until analysis.

Vervet monkey studies: As a pilot safety study, a single dose of Peptide 1 (SEQ ID NO. 1) at 3 mg/kg was administered to an adult male vervet/African green monkey (Chlorocebus aethiops sabaeus) via i.v. bolus. Serial blood samples were collected at 0.5, 1, 4, 8, 24, 48, 72, 96, 120, and 192 h post-administration.

Bioanalytical analysis: Clarified plasma samples from mice were directly diluted into 5% formic acid/5% acetonitrile. Quantitative analysis of Peptide 1 (SEQ ID NO. 1) was performed by LC-MS/MS with reverse-phase liquid chromatography (XBridge™ BEH phenyl 3.5 μm 3×100 mm column, Waters #186003328) on an Acquity™ H-Class UPLC (Waters) coupled to a Xevo™ TQ-S tandem electrospray mass spectrometry running MassLynx™ V4.1 (Waters). Quantitative mass spectroscopy was performed by multiple-reaction monitoring transition 417.32>517.21, with the area under the curve determined by TargetLynx™ (Waters). A synthetic theta defensin-like peptide was used as an internal standard (IS). The lower limit of quantification (LLOQ) of the assay was 1 ng/mL. Intra- and inter-assay precision (percent coefficient of variation [CV]) was ≤3%, and intra and interassay accuracy (% relative error) was ≤5%. Plasma Peptide 1 (SEQ ID NO. 1) concentration analyses for rats and cynomolgus monkey studies were performed at MicroConstants (San Diego, CA) by HPLC using a Mac-Mod™ Analytical ACE C4 column. The mobile phase was nebulized using heated nitrogen in Z-Spray source/interface set to electrospray positive ionization mode, and the compound was detected using MS/MS (LLOQ=10.0 ng/mL). Details of the method are summarized in the MicroConstants method No. MN20038. Clarified plasma samples from a serially sampled vervet were quantified by ultra-performance liquid chromatography Waters Acquity H-Class UPLC. The plasma samples were diluted directly (1:10) into 5% formic acid/5% acetonitrile and quantified by photodiode array (PDA AUC of 210 nm) using (C18 XBridge™ BEH 2.5 μm 2.1×150 mm Waters #186006709) running Empower software. Peptide 1 (SEQ ID NO. 1) peak and mass confirmation was performed on post PDA eluent using a Micromass Quattro Ultima™ mass spectrometer with MassLynx™ 4.1 (Waters). The lower limit of quantitation (LLOQ) ranged from 10-30 ng/mL (determined by sample background) to an upper limit of 50 ug/mL. RTD-2 (10 ug/mL) was used as an internal standard. Intra- and interassay precision (% coefficient of variation [CV]) was ≤3%, and intra and interassay accuracy (% relative error) was ≤5%.

Pharmacokinetic analysis: Non-compartmental analysis (NCA) was performed using Phoenix WinNonlin™ (version 8.3.1, Certara USA, Inc.; Princeton NJ) to determine the PK parameters in mice, rats, cynomolgus monkeys, and the vervet. Nominal sampling times were used in the analysis, and data below the lower limit of quantification of the assay were excluded from the analysis. The maximum plasma concentration (Cmax) was determined from visual inspection of the data. In addition, the following parameters were calculated: terminal elimination rate constant (λz), area under the curve extrapolated to infinity (AUC0-∞), area under the curve to dosing interval (AUCτ), mean residence time (MRT), clearance (CL), and volume of distribution at steady state (Vss). AUC was calculated using the linear up, log down method, and the λz was calculated using up to the last four data points of the log-linear terminal phase of the concentration-time profile. Due to sparsely sampled data in mice and rats, the sparse sampling calculation methodology in Phoenix WinNonlin™ was used, which generated a single estimate without standard error for all parameters, except for Cmax. For rats and cynomolgus monkeys, all parameters were calculated using sampling times relative to the beginning of the i.v. infusion.

Dose proportionality: Dose proportionality of Cmax and AUC_{0-∞} was evaluated in cynomolgus monkeys administered a single i.v. dose of Peptide 1 (SEQ ID NO. 1) ranging from 0.3 to 15 mg/kg using a natural log-transformed power model. Dose proportionality was concluded if the slope and the corresponding 95% confidence interval of the linear regression included.

Interspecies allometric scaling: Single-dose PK data from mice, rats, cynomolgus monkeys, and a vervet was used to predict human PK parameters using simple allometry. The relationship between CL or Vss obtained from the NCA, and the body weight was described using the following equation: Y=a·BW^{b}, where Y is the PK parameter (e.g., CL or Vss), BW is the bodyweight of the species, a is an allometric coefficient, and b is an allometric exponent. Linear regression was performed on log-transformed data. The predicted human equivalent dose was calculated based on the allometrically scaled clearance using the following equation: Dose=CL*AUC_{0-∞}. The corresponding average AUCτ and AUC_{0-∞} at NOAEL and LOAEL, respectively, were used to convert the doses at NOAEL and LOAEL in preclinical animals to HED.

Biodistribution [¹⁴C]-radiolabeled Peptide 1 (SEQ ID NO. 1) in female rats: Five SD rats (195 and 200 g b.w.) with jugular vein catheters (JVC) were each injected with 200 μL of 5 mg/mL Peptide 1 (SEQ ID NO. 1) in saline containing ˜4 million CPM of [¹⁴C]-Peptide 1. The ¹⁴C-Peptide 1 (SEQ ID NO. 1) was created by substituting the natural glycine residue on position 1 of the cyclic peptide with a ¹⁴C labeled glycine. The JVC line was cleaned with 70% isopropyl alcohol, and the line plug was removed. A new 25 G blunt needle with a 1 mL syringe containing injectable solutions was used for each injection. The JVC line was first cleared with 100 μL saline, followed by the Peptide 1 (SEQ ID NO. 1) solution, then cleared with an additional 100 μL of saline. Tissues and organs were harvested into separated vials and weighed. Small organs such as lymph nodes, kidneys, and heart/lungs were processed whole. Large organs (e.g., liver, muscles, subcutaneous fat pads) were sampled from representative areas or those of interest (e.g., subcutis at the injection site). Organs were then dissolved in 2 mL Solvable™ (Perkin Elmer 6NE9100) for up to 1 g of tissue. For a large section of skin and other organs, 4 or 6 mL were added. Vials were incubated in a 60° C. water bath for 18-22 hours, then removed and allowed to cool to room temperature. Two mL of dissolved tissue were added to a fresh scintillation tube for color correction with 100 μL of 0.1 M EDTA and 2×100 μl 30% hydrogen peroxide. Samples were allowed to stand at room temperature for 1 hour, incubated in a 37° C. incubator for 1 hour, then incubated in a 60° C. water bath for 1 hour. If necessary to prevent boiling over, samples were removed from the heat source temporarily before continuing. Samples were cooled before 10 mL of Ultima Gold (Perkin Elmer 6013321) were added to each vial, then contents were mixed and allowed to stand in the dark at 22° C. for 1 h. Scintillation counting was an average of 2×1-minute counts using a Packard Tri-Card™ 2100TR scintillation counter. Urine was collected over 1 h or 24 h after i.v. infusion and 500 μL was added to a scintillation vial processed as described above for scintillation counting. The stomach and its content were dissolved with 6 mL of Solvable™ and processed as other tissues. The duodenum, jejunum, and ileum were flushed with saline to remove luminal contents then the tissues were processed with Solvable™ as described above. The large intestine was opened lengthwise to remove fecal content and rinsed with saline to remove remaining luminal contents, then processed as described above. Feces were collected, transferred into a 500 mL plastic cup, and treated with 50 mL of 7% sodium hypochlorite and allowed to react for 1 hour at 22° C. followed by 1 h in a 60° C. water bath. Two ml of the resulting suspension were transferred to a scintillation vial and mixed with 10 mL of scintillation fluid. Contents were mixed and allowed to stand for 1 hour before scintillation counting.

### Safety

Evaluation of safety in rats and cynomolgus monkeys was based on clinical observations, survival, body weight, food consumption, clinical pathology (hematology, clinical chemistry, and coagulation), urinalysis, ophthalmology, macroscopic findings at necropsy, and microscopic histopathology. The schedule of assessments for these studies is summarized in Table 10.

### Statistical Analysis

Statistical analysis of the PK data was performed with GraphPad Prism™ version 9.1.2 (GraphPad Software, Inc., San Diego, CA). Shapiro-Wilk test was used to check for normality. One-way ANOVA with Bonferroni's multiple comparisons test was performed to compare Cmax across doses (5-, 10- and 20 mg/kg) in rats, and λz, AUC, Cmax, CL, and Vss across doses (5-, 10- and 15 mg/kg) in cynomolgus monkeys. Unpaired t-test was used to compare PK parameters (λz, AUC, Cmax, CL, and Vss) between different days (Days 1 vs. 10) within each dosing group and between sexes. Statistical analyses of safety data were performed using SAS 9.4, considering a 95% statistical significance. Differences in body weight, change in body weight, and clinical pathology (hematology, serum chemistry, coagulation) were compared between each dose group using Kruskal Wallis with Dunn's multiple comparison test. The data analysis was performed independently for each sex.

The above safety studies indicate that cyclic peptides such as Peptide 1 (SEQ ID NO. 1) can be safely systemically administered in amounts of up to 1 mg/kg, 2.5 mg/kg, 5 mg/kg, 7.5 mg/kg, 10 mg/kg, 12.5 mg/kg, 15 mg/kg, 17.5 mg/kg, or 20 mg/kg. While such studies have focused on Peptide 1 (SEQ ID NO. 1), it is contemplated that the findings can be extended to other, similar peptides. Such peptides include but are not limited to Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16).

### Example 3: Anti-Sars-CoV-2 Activities of Naturally Occurring Cyclic Peptides

The antiviral, antiprotease, and anti-inflammatory properties of certain cyclic peptides were tested in the assays described below.

SARS, MERS, and COVID-19 are human diseases caused by coronaviruses. Coronaviruses cause disease by binding to receptors on host cells, proteolytic processing of the binding complex, internalization of the virus, intracellular replication of the virus, cell death, viral spread to adjacent cells and other tissues, and dysregulated inflammation mediated by cytokines and chemokines.

The host cell proteases that have been implicated in SARS-CoV-2 include three different protease classes: TMPRSS2 (serine protease), cathepsin B/L (cysteine protease), and furin (metalloprotease). naturally occurring cyclic peptides such as Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16 can inhibit metalloproteases and cysteine proteases and in this regard provide a unique cross class anti-protease activity. Inventors believe that such naturally occurring cyclic peptides can also inhibit serine proteases. Further, Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16) can, suppress pathologic inflammation characteristic of COVID19 by inhibiting the expression and/or release of proinflammatory cytokines in vitro and in vivo.

The data provided herein shows that naturally occurring cyclic peptides such as can be effective in the treatment of coronavirus (e.g., SARS-CoV-2) infection.

### Materials and Methods

Antiviral assay. Calu3 2B4 cells were grown in EMEM medium containing 20% FBS and 1% Penicillin/Streptomycin (complete EMEM). Cells were trypsinized and resuspended in complete EMEM medium at 5×10⁵ cells/mL and 1 mL of cells were added to wells of a 12 well plate. After growing cells overnight, the medium was removed and 1 mL EMEM medium containing 1% FBS and Pen/Strep (1% FBS-EMEM medium) was added to the cells. The cells were then transferred to BSL3 facility and incubated for 2 h at 37° C., 5% CO₂. The medium was removed and 1% FBS-EMEM containing either vehicle (0.01% acetic acid) or peptide along with SARS-CoV-2 viral stock (0.1 to 0.2 MOI) was added to the cells. The activities of naturally occurring cyclic peptides Peptide 1 (SEQ ID NO. 1), Peptide 2 (SEQ ID NO. 2), and Peptide 5 (SEQ ID NO. 5) were characterized. Assays were performed in triplicate and for non-infection control, viral stock was substituted with 1% FBS-EMEM medium. Cells were incubated for 24 h, the medium was removed and Trizol was added to the cells.

RNA was isolated from the Trizol solutions using the Direct Zol™ RNA Miniprep kit (Zymo Research, Irvine, CA). The concentration of RNA was analyzed by Nanodrop 2000 and RNAs that had a low concentration or an A260/A280 of less than 1.8 were purified on RNA Clean and Concentrator 5 kit (Zymo Research) and resolved on 0.8% agarose TAE gel to confirm RNA integrity. Approximately 200 ng RNA was used for making first strand cDNA using the RT2 First Strand™ cDNA kit (Qiagen) as per manufacturer. Real time quantitative polymerase chain reaction (RT-qPCR) was performed using the RT2 Sybr green mastermix (Qiagen) using primers shown in Table 1. The conditions for RT-qPCR were 1 cycle 95° C. 10 min and 40 cycles of 95° C. 15 secs, 60° C. 1 min. A melt curve was performed to confirm formation of a single product.

All cDNAs were initially probed using human gDNA primer to confirm lack of genomic DNA (Ct>35) and using RTC primer to confirm similar reverse transcription efficiency. Data was analyzed using BioRad Maestro™ software. The 2^{-ΔΔCt} method was used to calculate fold change and dCt values were used for statistical analysis, P<0.05 was considered significant.

TABLE 11
SARS-CoV-2 Nucleocapsid phosphoprotein
(N) gene Primers

| | | |
|---|---|---|
| ATGCTGCAATCGTGCTACAA | Forward | SEQ ID NO. 17 |
| CCTCTGCTCCCTTCTGCGTA | Reverse | SEQ ID NO. 18 |

Viral replication was quantified using SARS-CoV-2 nucleocapsid gene (N) expression relative to actin B gene (ACTB) gene expression using the 2^{-ΔΔCt} method. Change of dCt values were used for statistical analysis. Similar anti-viral activities were obtained using the RNA polymerase P subunit 30 (RPP30) as the internal control

TMPRSS2 inhibition assays. Peptides (0-50 μg/ml) were tested for their inhibitory activities against TMPRSS2 in cellular assays using Vero E6 cells, Caco-2 cells, or Calu-3 cells using standard conditions. TMPRSS2 activity was assayed in real time for 120 min by monitoring the cleavage of the TMPRSS2 substrate, Boc-Leu-Gly-Arg-AMC. Following the 2 h enzyme assay, cells were evaluated for viability using the CellTiter-Glo cellular toxicity assay. No cytotoxicity was observed for any of the five peptides at >10 μg/mL.

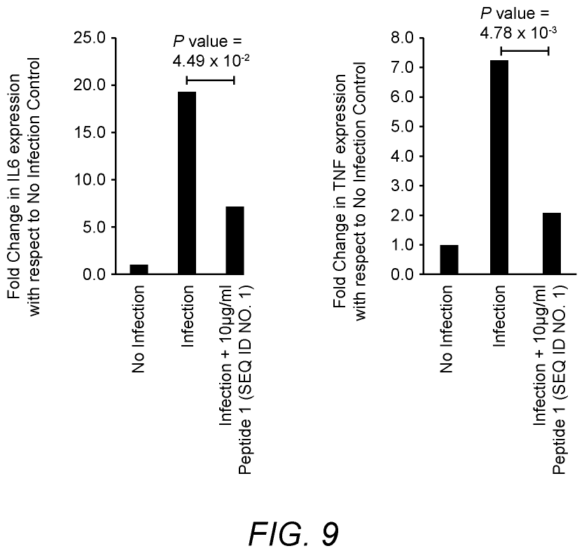

TNF and IL-6 gene expression in virally infected Calu3 2B4 cells. Calu3 2B4 lung epithelial (Calu3) cells were infected with SARS-CoV-2, co-incubated with 0-10 μg/mL of Peptide 1 (SEQ ID NO. 1) and analyzed for peptide effect on mRNA expression of TNF, IL-6, and GAPDH (control). Results shown in FIG. 9.

Results

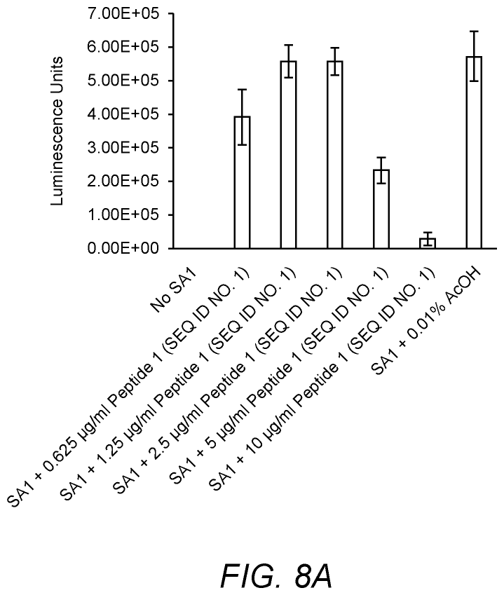

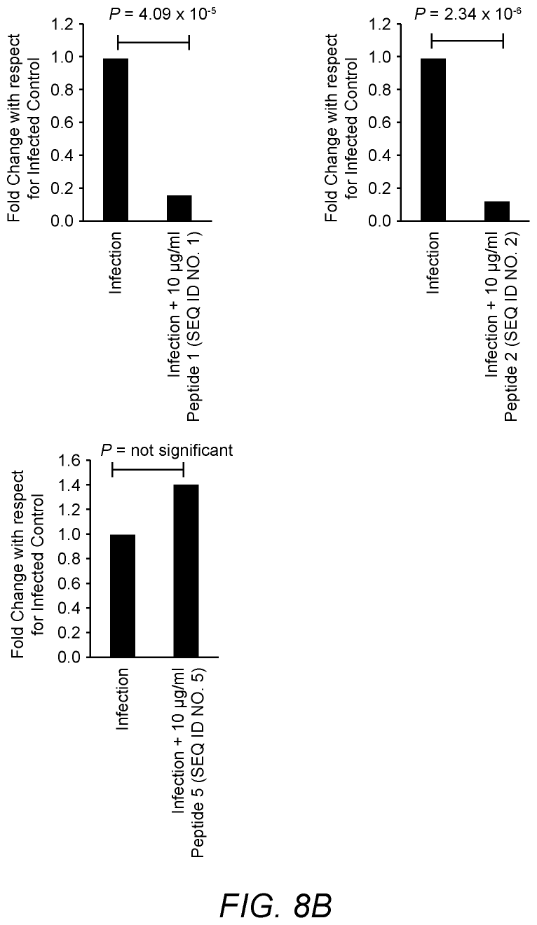

Anti-SARS-CoV-2 properties of naturally occurring cyclic peptides. As shown in FIG. 8A, Peptide 1 (SEQ ID NO. 1) and Peptide 2 (SEQ ID NO. 2) were highly effective in blocking viral replication in Calu3 2B4 cells, human bronchial epithelial cells that are routinely used to assess viral infection of SARS coronaviruses. The antiviral effects of Peptide 1 (SEQ ID NO. 1) and Peptide 2 (SEQ ID NO. 2) were highly significant (P<0.0001 in each case). While Peptide 5 (SEQ ID NO. 5) had no apparent antiviral effect at the concentrations used, Inventors believe such effects may be evident upon further optimization. As shown in FIG. 8A, both peptides were highly effective in blocking viral replication (P<0.001 in each case).

As shown in FIG. 9, SARS-CoV-2 infection markedly increased TNF and IL-6 mRNA expression, but not GAPDH. Peptide 1 (SEQ ID NO. 1) selectively inhibited expression of both cytokines in virus infected cells in a concentration dependent manner but had no effect on GAPDH.

Total RNA isolated from Calu3 cells was reverse transcribed and real time PCR was performed using SARS-CoV-2 virus Nsp14 gene primers. Peptide 1 (SEQ ID NO. 1) inhibited viral replication, as measured by Nsp14 gene expression, in a concentration dependent manner. Nsp14 specific PCR product was not observed in either the no-RNA control (data not shown).

Without wishing to be bound by theory, it is contemplated that anti-viral effects of naturally occurring cyclic peptides are a result of inhibition of proteases necessary for entry of coronavirus into a cell, intracellular inhibition of viral replication by naturally occurring cyclic peptide that has entered the cell, and/or a combination of these effects.

Cellular TMPRSS2 inhibition. TMPRSS2 is a cell surface serine protease that has been shown to be essential for the processing of the viral spike protein and internalization of SARS-CoV-2. Preliminary experiments demonstrated that Peptide 1 (SEQ ID NO. 1) concentration-dependently inhibited TMPRSS2 expressed on the surface of Caco-2 cells and Calu3 cells, with an IC50 of 6.5-8 μg/mL enzyme inhibition in both cell lines.

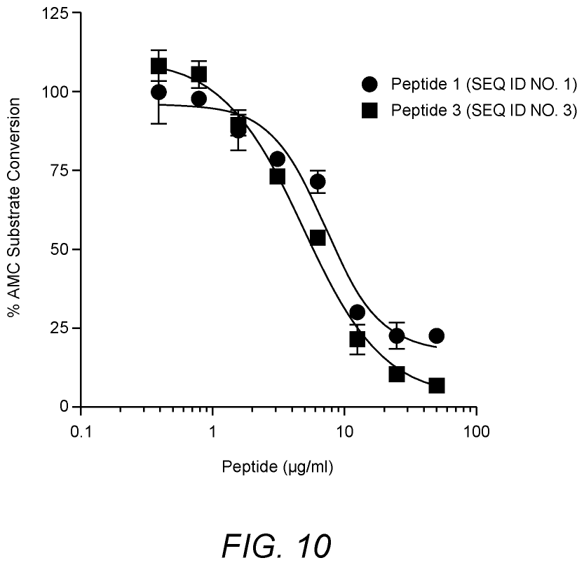

Peptide 1 inhibits TMPRSS2. TMPRSS2 is a cell surface protease required for infection of host cells by SARS-CoV-2. Inhibition of TMPRSS2 activity has been shown to inhibit SARS-CoV-2 infection of susceptible cells. Peptide 1 (SEQ ID NO. 1) and other naturally occurring cyclic peptides were tested for TMPRSS2 inhibitory activity using Caco-2 and Calu3 cells, both of which express surface TMPRSS2. As shown in FIG. 10, Peptide 1 (SEQ ID NO. 1) inhibited TMPRSS2 expressed Caco-2 cells in a concentration dependent manner. Inhibition of TMPRSS2 by Peptide 3 (SEQ ID NO. 3) was higher than Peptide 1 (SEQ ID NO. 1) at higher peptide concentrations. Both peptides similarly inhibited TMPRSS2 expressed by Calu3 cells.

Of note, Peptide 5 (SEQ ID NO. 5) had no TMPRSS2 inhibitory activity at the highest concentration tested (50 μg/mL). This is significant in that Peptide 5 is also inactive in the viral replication assay.

Identification of naturally occurring cyclic peptides with superior TMPRSS2 inhibitory activity. Cell-based assays as described above utilizing Caco-2 and Calu3 cells were employed to determine the IC₅₀'s of naturally occurring cyclic peptides. Briefly, Caco-2 cells (top panel) or Calu3 cells (bottom panel) were incubated with 0-50 μg/mL of the indicated peptide in the presence of TMPRSS2 substrate as follows. Vero cells were infected with 160 TCID₅₀ of SA1 virus in DMEM containing 1.5% FBS in 96 well plates, and the indicated concentrations of peptide. After 24 h incubation at 37° C., viral titer was quantified by luciferase assay and compared with no SA1 control, solvent (0.01% AcOH). No SA1 is the no virus control. Substrate conversion was monitored in a fluorescence plate reader for 2 h and inhibition of enzyme activity was expressed relative to solvent control. A number of the peptides were more potent than Peptide 1 in one or both cell-based assays, identifying naturally occurring cyclic peptides with superior anti-protease activities.

Peptide 1 (SEQ ID NO. 1) suppresses expression of proinflammatory cytokines by SARS-CoV-2 infected Calu3 cells. Infection of Calu3 and normal human bronchial epithelial cells with SARS-CoV-2 has been shown to increase expression of TNF and IL-6 genes, driving dysregulated inflammation in COVID-19. Calu3 2B4 lung epithelial (Calu3) cells were infected with SARS-CoV-2, co-incubated with 0-10 μg/mL of Peptide 1 (SEQ ID NO. 1), or vehicle for 24 h at 37° C. in 5% CO₂ in DMEM+1% FBS, medium was removed, and mRNA expression analyzed by RT-PCR normalized to ACTB expression. for peptide effect on mRNA expression of TNF, IL-6, and GAPDH (control). Viral infection markedly increased TNF and IL-6 mRNA expression, but not GAPDH. Peptide 1 (SEQ ID NO. 1) inhibited expression of both cytokines in virus infected cells in a concentration dependent manner but had no effect on GAPDH.

### Summary

Naturally occurring cyclic peptides are described that inhibit SARS-CoV-2 replication under conditions where the peptide is non-toxic to cells. Inhibition of viral replication correlates with the ability of antiviral peptides to inhibit TMPRSS2 enzymatic activity. In addition, at least one peptide also suppresses expression of proinflammatory genes that are implicated in the severity of COVID pneumonia.

### Example 4: Peptide 1 (SEQ ID NO. 1) Inhibits Infection of Vero E6 Cells by SARS-CoV-2 S-Typed Pseudovirus (SA1)

It has been found that Peptide 1 (SEQ ID NO. 1) is effective in inhibiting growth of a SARS-CoV-2 analog in cells when applied following exposure. As shown in FIG. 8A, Vero E6 cells (which are susceptible to coronavirus) show reduced growth of a SARS-CoV-2 pseudovirus (SA1 virus) when treated with Peptide 1. In these studies, Vero E6 cells were infected with 160 TCID₅₀ of SARS-CoV-2 pseudovirus (SA1) in DMEM containing 1.5% FBS in 96 well plates, and the indicated concentrations of Peptide 1. After 24 hours incubation at 37° C., viral titer was quantified by luciferase assay and compared with no SA1 control, solvent (0.01% AcOH), and no virus controls. No SA1 is the no virus control.

As shown, Peptide 1 (SEQ ID NO. 1) inhibits SA1 infection of the Vero cells in a concentration dependent manner. It is contemplated that other naturally occurring cyclic peptides (e.g., Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), and/or Peptide 16 (SEQ ID NO. 16)) can provide similar effects. Without wishing to be bound by theory, it is contemplated that anti-viral effects of naturally occurring cyclic peptides are a result of inhibition of proteases necessary for entry of coronavirus into a cell, intracellular inhibition of viral replication by naturally occurring cyclic peptide that has entered the cell, and/or a combination of these effects.

In some embodiments two or more naturally occurring cyclic peptides can be administered simultaneously. In such embodiments the naturally occurring cyclic peptides used can be provided in any suitable ratio. For example, a mixture of Peptide 1 (SEQ ID NO. 1) and Peptide 2 (SEQ ID NO. 2) can be administered in a Peptide 1:Peptide 2 ratio ranging from 100:1 to 1:100, or any intermediate value. In such embodiments the selection of naturally occurring cyclic peptides and/or the ratio between the naturally occurring cyclic peptides can be adjusted during the course of treatment. Such adjustment can be made on the basis of symptoms, viral titer, and/or historical data.

In some embodiments of the inventive concept, compositions and methods as described above can be implemented as part of cotherapy with conventional anti-viral therapy. For example, treatment with naturally occurring cyclic peptides can be carried out in combination with antibodies directed to portions of the SARS-CoV-2 virus. Such antibodies can be polyclonal (e.g., immunoglobulins of convalescent serum) or monoclonal. Examples of suitable monoclonal antibodies include bamlanivimab, etesevimab, casirivimaband, and/or sotrovimab. Naturally occurring cyclic peptides can also be used in combination with small molecule (i.e., molecular weight of less than 1 kD) drugs directed to SARS-CoV-2. Examples of suitable small molecule drugs include remdesivir.

In some embodiments of the inventive concept compositions and methods as described above can be implemented as part of cotherapy with one or more therapies to enhance or improve response of the innate immune system to viral infection. Examples include cotherapy with azithromycin, CSY0073, and Mac26.

Inventors contemplate that, in utilizing safely administrable compounds with a broad range activities and in addressing fundamental processes common to entry and processing of coronaviruses as well as inflammatory sequelae of coronavirus infection, compositions and methods described herein are effective against various strains of SARS-CoV-2 (e.g., delta, lambda, omicron, etc.). Similarly, Inventors believe that compositions and methods as described herein will remain effective against new strains and/or variants of SARS-CoV-2 as they develop and can be effective both treatment of disease and in limiting the spread of such strains and/or variants as specific vaccines are developed.

It should be apparent to those skilled in the art that many more modifications besides those already described are possible without departing from the concepts disclosed herein. The disclosed subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1-53. (canceled)

54. A method of treating an individual for infection by a coronavirus, comprising:
determining that the individual is in need of treatment of infection by the coronavirus; and
administering a formulation comprising a first protease inhibitor activity and an anti-inflammatory activity.

55. The method of claim 54, wherein the coronavirus is SARS-CoV-2.

56. The method of claim 54, wherein the first protease activity comprises at least one of a serine protease inhibitor activity, a cysteine protease inhibitor activity, and a metalloprotease activity.

57. The method of claim 54, wherein the first protease activity comprises a serine protease inhibitor activity, a cysteine protease inhibitor activity, and a metalloprotease inhibitor activity.

58. The method of claim 54, wherein the first protease inhibitor activity is selected from the group consisting of inhibition of TMPRSS2, inhibition of Cathepsin B/L (CatB/L), and inhibition of furin.

59. The method of claim 54, wherein the first protease activity and the anti-inflammatory activity are embodied in a single molecular species.

60. The method of claim 54, wherein the formulation further comprises a second protease inhibitor activity, and wherein the anti-inflammatory activity comprises the second protease inhibitor activity.

61. The method of claim 60, wherein the second protease inhibitor activity reduces processing of a pro-inflammatory cytokine.

62. The method of claim 60, wherein the second protease inhibitor activity inhibits a sheddase.

63. The method of claim 54, wherein the formulation comprises a naturally occurring cyclic peptide exhibiting both the first protease inhibitor activity and the anti-inflammatory activity.

64. The method of claim 63, wherein the naturally occurring cyclic peptide is selected from the group consisting of Peptide 1 (SEQ ID NO. 1), Peptide 2 (SEQ ID NO. 2), Peptide 3 (SEQ ID NO. 3), Peptide 4 (SEQ ID NO. 4), Peptide 5 (SEQ ID NO. 5), Peptide 6 (SEQ ID NO. 6), Peptide 7 (SEQ ID NO. 7), Peptide 8 (SEQ ID NO. 8), Peptide 9 (SEQ ID NO. 9), Peptide 10 (SEQ ID NO. 10), Peptide 11 (SEQ ID NO. 11), Peptide 12 (SEQ ID NO. 12), Peptide 13 (SEQ ID NO. 13), Peptide 14 (SEQ ID NO. 14), Peptide 15 (SEQ ID NO. 15), Peptide 16 (SEQ ID NO. 16),

65. The method of claim 54, wherein administration comprises systemic administration of the formulation.

66. The method of claim 65, wherein systemic administration comprises intravenous administration of the formulation.

67. The method of claim 66, wherein intravenous administration comprises injection to provide a naturally occurring cyclic peptide or a synthetic cyclic peptide in an amount sufficient to provide more than 5 mg/kg.

68. The method of claim 67, wherein intravenous administration comprises injection to provide a naturally occurring cyclic peptide or a synthetic cyclic peptide in an amount sufficient to provide less than or equal to 20 mg/kg.

69. The method of claim 54, wherein the formulation is provided as a droplet suspension, and wherein administration comprises inhalation of the droplet suspension.

70. The method of claim 54, comprising coadministration of the formulation with an antibody directed to the coronavirus.

71. The method of claim 19, wherein the antibody is selected from the group consisting of a convalescent serum and a therapeutic monoclonal antibody directed to the coronavirus.

72. The method of claim 54, comprising coadministration of a small molecule drug that is effective against the coronavirus, wherein the small molecule drug has a molecular weight of less than 1 kD.
